(19) United States
(12) Patent Application Publication
(10) Pub. No.: US 2025/0283030 A1
(43) Pub. Date: Sep. 11, 2025

(54) Biosynthesis of Phenylpropanoid Compounds

(71) Applicant: BGENE GENETICS, Grenoble (FR)

(72) Inventors: William Merré, Grenoble (FR); Ricardo de Andrade, Grenoble (FR); Caroline Ranquet, Grenoble (FR)

(73) Assignee: BGENE GENETICS, Grenoble (FR)

(21) Appl. No.: 18/289,895

(22) PCT Filed: May 9, 2022
(86) PCT No.: PCT/FR2022/050880

§ 371 Date: Nov. 8, 2023

(30) Foreign Application Priority Data
May 12, 2021 (FR) 2105009

(51) Int. Cl. C12N 1/20; C12N 9/00; C12N 9/02; C12N 9/10; C12N 9/88; C12N 15/78; C12P 7/26; C12P 7/42; C12P 17/06; C12P 19/32; C12R 1/40

(52) U.S. Cl. CPC C12N 1/20; C12N 9/0071; C12N 9/1029; C12N 9/1085; C12N 9/88; C12N 9/93; C12N 15/78; C12P 7/26; C12P 7/42; C12P 17/06; C12P 19/32; C12R 2001/40; C12Y 114/16001; C12Y 205/01054; C12Y 402/01096; C12Y 403/01023; C12Y 602/01012

(57) ABSTRACT

The present invention relates to the field of the production of phenylpropanoid compounds, especially that of genetically modified strains for the production of phenylpropanoid compounds. In particular, the invention relates to a genetically modified strain of Pseudomonas putida comprising a mutated AroF-I gene encoding 3-deoxy-D-arabino-heptulosonate-7-phosphate (DAHP), and to the use thereof for the synthesis of phenylpropanoid compounds, in particular coumaric acid or frambinone.

### TECHNICAL FIELD

The present invention relates to the field of the production of phenylpropanoid compounds, and particularly to genetically modified strains for the production of phenylpropanoid compounds, such as coumaric acid or frambinone.

### PRIOR ART

For many years, the bioproduction of “natural” flavorings and fragrances has been an important area of research for industry, in order to meet the needs of consumers who seek to be increasingly environmentally responsible. The biology of synthesis, particularly using microorganisms, makes this natural production possible, but yields are not always sufficient for large-scale production.

The flavor of raspberries (Rubus idaeus) is associated with more than 200 compounds, but frambinone, a natural phenolic compounds, is the compound with the greatest impact, defining the characteristic raspberry flavor (Klesk et al., 2004, J. Agric. Food Chem. 52, 5155-61; Larsen et al., 1991, Acta Agric. Scand. 41, 447-54).

Since it is only present in small amounts in raspberries (1-4 mg per kg of fruit), natural frambinone is extremely valuable (Larsen et al., 1991). However, because the natural availability thereof is limited, producing it by means of biotechnology is highly desirable.

In this context, the biosynthesis pathway of phenylpropanoid compounds, particularly frambinone, can be recreated within a microorganism by inserting heterologous genes encoding some of the key enzymes of this pathway.

Tyrosine is an important amino acid for the biosynthesis of phenylpropanoid compounds because it is in particular the precursor of coumaric acid or of frambinone.

Indeed, frambinone can be obtained from the aromatic amino acid L-tyrosine as initial substrate, via a 4-step biosynthesis pathway.

In the first step, the tyrosine is deaminated by a tyrosine ammonia lyase (TAL, EC 4.3.1.23) to form coumaric acid. Catalyzed by a 4-coumarate:CoA ligase (4CL, EC 6.2.1.12), a molecule of Coenzyme A (CoA) is grafted onto the coumaric acid. The coumaroyl-CoA is then converted by a benzalacetone synthase (BAS, EC 2.3.1.212) into 4-hydroxybenzalacetone. This reaction is a decarboxylative condensation, and uses a unit of malonyl-CoA as co-substrate. The final step is the reduction of the 4-hydroxybenzalacetone into frambinone by a benzalacetone reductase.

Thus, the development of strains that overproduce tyrosine as a platform for the production of phenylpropanoid compounds, particularly frambinone, is a crucial starting point for the biosynthesis of these compounds.

However, the production of tyrosine in microorganisms is as complex as it is closely regulated. This is because some enzymes in the tyrosine production pathway, also known as the shikimate pathway, are regulated in a negative feedback loop by this amino acid, which inhibits their activity by bonding to an inhibition site when the concentration in the cell becomes too high. This system of regulation, when too great a quantity of tyrosine is present in the microorganism, is referred to as feedback inhibition.

In spite of this, it should be possible, by mutagenesis, to obtain enzymes that are “resistant” to this inhibition and therefore to deregulate the tyrosine production pathway. These mutants, which are resistant to tyrosine feedback inhibition, are referred to as fbr mutants, for “feedback resistant”.

Numerous articles describe the deregulation of this pathway in E. coli, and one enzyme has been particularly well studied: DAHP synthase (DAHP=3-deoxy-D-arabino-heptulosonate-7-phosphate). The first reaction in the shikimate pathway consists of the condensation of a phospho(enol)pyruvate (PEP) and an erthythrose-4-phosphate (E4P) to give DAHP.

This DAHP synthase activity is carried out by 3 isoenzymes in E. coli: AroG (feedback-inhibited by phenylalanine), AroF (feedback-inhibited by tyrosine) and AroH (feedback-inhibited by tryptophan). The structures of these proteins are known, and the substructures (and also the amino acids involved) associated with feedback inhibition are also thoroughly described in the literature.

Kikuchi et al., 1997 and Cui et al., 2019, identified, described and studied, in E. coli, the mutants AroGfbr and AroFfbr which are resistant to phenylalanine and tyrosine feedback inhibition, respectively. Fbr mutants of the protein TyrA (mutant tyrAfbr) have also been described in E. coli by Lutke-Eversloh and Stephanopoulos, (2005) (Lutke-Eversloh and Stephanopoulos, Appl. Environ Microbiol. 2005 November; 71(11): 7224-8).

In 2007, Lutke-Eversloh and Stephanopoulos (Lutke-Eversloh and Stephanopoulos, Appl. Environ Microbiol. 2007 November; 75(1): 103-10) also described E. coli strains that overproduce tyrosine, obtained by combining these various mutated enzymes. These strains were used to produce phenylpropanoids such as coumaric acid (Kang et al., 2012), or naringenin, particularly via mutation of the rpoA gene in addition to the aroGfbr and tyrAfbr mutations (Santos et al., 2011).

A review of the modifications carried out to obtain E. coli strains that overproduce tyrosine is described in the article “Modular engineering of L-tyrosine production in E. coli” (Juminaga et al., 2011).

Phenylpropanoid compounds have also been synthesized from Saccharomyces cerevisiae strains that overexpress tyrosine, as described by Rodriguez et al., 2015.

Document GB 2 416 769 describes the possibility of producing frambinone using microorganisms containing genes encoding the enzymes 4CL and BAS, at least one of which comes from a heterologous source. The preferred microorganism is E. coli (strain BL21) and may further comprise a sequence encoding BAR, C4H, PAL and/or CHS, with the sequence encoding BAR advantageously being endogenous.

However, it appears that E. coli or S. cerevisiae do not have good tolerance for the toxicity of phenylpropanoid compounds, and are therefore not the most well-suited microorganisms for the production thereof.

Bacteria of the genus Pseudomonas are more tolerant to these highly toxic molecules, particularly the bacterium Pseudomonas putida (Calero et al., 2017). However, the enzymes involved in the production of aromatic amino acids in P. putida have not been described in detail.

Consequently, the overproduction of shikimate pathway derivatives such as tyrosine in P. putida involves the use of AroGfbr and TyrAfbr mutants originating from E. coli, which is not particularly effective (Calero et al., 2016).

The overproduction of tyrosine and phenol has been described in the bacterium Pseudomonas taiwanensis VLB120 by implementing point mutations in 3 genes: trpEP290S, aroF-I P148L and pheAT3101 (Wynands et al., Metab Eng. 2018 May, 47:121-133).

However, there is still a need to develop novel enzymes and novel strains of Pseudomonas putida that overproduce tyrosine, enabling the production of phenylpropanoid compounds, particularly coumaric acid and frambinone.

### Technical Problem

The strains that overproduce tyrosine that have been developed to date are essentially E. coli and S. Cerevisiae strains. However, these strains do not have good tolerance for the toxicity of phenylpropanoid compounds, and are therefore not the most well-suited microorganisms for the production thereof.

Thus, there is a particular need to develop novel strains that overproduce tyrosine, enabling the production of phenylpropanoid compounds.

The present disclosure will improve the situation.

### SUMMARY

One of the aspects of the present invention relates to a genetically modified strain of Pseudomonas putida, comprising a mutated AroF-I gene encoding 3-deoxy-D-arabino-heptulosonate-7-phosphate (DAHP) synthase, having the sequence SEQ ID NO:1 and having at least one P160L mutation, a P160L/Q164A double mutation, or a P160L/S193A double mutation, preferably a P160L/S193A double mutation.

Another aspect of the invention relates to a method for synthesizing a phenylpropanoid compound or a phenylpropanoid derivative by employing a genetically modified strain of Pseudomonas putida according to the invention.

Finally, the invention also relates to the use of a genetically modified strain of Pseudomonas putida for the synthesis of phenylpropanoid compounds.

The characteristics set out in the following paragraphs can optionally be implemented. They may be implemented independently or in combination with one another:

### BRIEF DESCRIPTION OF THE FIGURES

Other features, details and advantages will become apparent upon reading the following detailed description and upon examining the appended drawings, in which:

FIG. 1 Graph of PEP consumed, in mM, in the presence or absence of aromatic amino acid, by the wild-type (WT) AroF-I protein. The results presented are from three independent replicas.

FIG. 2 Graph showing the share of PEP consumed, in mM, in the presence or absence of aromatic amino acid, by the AroF-I WT protein compared to the single mutants AroF-I-G191, AroF-I-P160 and AroF-I-S193. The results presented are from three independent replicas.

FIG. 3 Graph showing the share of PEP consumed, in mM, in the presence or absence of aromatic amino acid, by the AroF-I WT protein compared to the double mutants AroF-I-P160_G191, AroF-I-P160L_Q164, AroF-I-P160_S190 and AroF-I-P160_S193. The results presented are from three independent replicas.

FIG. 4 Graph showing the share of PEP consumed, in mM, in the presence or absence of aromatic amino acid, by the AroF-I WT protein compared to the single mutants AroF-I-P160 and AroF-I-S193 and to the double mutant AroF-I-P160_S193. The results presented are from three independent replicas.

FIG. 5 presents the conjugation protocol that can be used to transform and genetically modify P. putida.

FIG. 6 Graph presenting the production of coumaric acid (PCA) and cinnamic acid (CA) by Pseudomonas putida strains expressing the enzymes AroF-I WT/TAL (aroF-I WT) and aroF-I fbr P160L/S193A/TAL (aroF-I fbr).

FIG. 7 Graph presenting the production of total phenolics and the proportion of coumaric acid in the total phenolics (% PCA) by Pseudomonas putida strains expressing the enzymes enzymes AroF-I WT/TAL (aroF-I WT), AroF-I WT/TAL+empty plasmid (aroF-I WT+control), AroF-I WT/TAL+phhA/B plasmid (aroF-I WT+phhA/B), aroF-I fbr P160L/S193A/TAL (aroF-I fbr), aroF-I fbr P160L/S193A/TAL+empty plasmid (aroF-I fbr+control), and aroF-I fbr P160L/S193A/TAL+phhA/B plasmid (aroF-I fbr+phhA/B). Expression of the phhA/B genes from the araC/pBAD promoter is induced or non-induced.

### DESCRIPTION OF THE EMBODIMENTS

A first subject of the present invention therefore relates to a genetically modified strain of Pseudomonas putida, comprising a mutated AroF-I gene encoding 3-deoxy-D-arabino-heptulosonate-7-phosphate (DAHP) synthase, the sequence of which has at least 80% identity to the sequence SEQ ID NO:1, and having at least one P160L mutation, a P160L/Q164A double mutation, or a P160L/S193A double mutation, preferably a P160L/S193A double mutation.

For the purposes of the present description, the expressions “genetically modified strain of Pseudomonas putida”, “modified strain of Pseudomonas putida”, “genetically modified strain” and “modified strain” are considered to be synonymous.

In particular, “genetically modified strain” means a strain which comprises either (i) at least one recombinant nucleic acid, or transgene, integrated stably into the genome thereof and/or present on a vector, for example a plasmid vector, or (ii) one or more non-natural mutations by nucleotide insertion, substitution or deletion, said mutations being obtained by transformation techniques or gene editing techniques known to those skilled in the art.

Indeed, techniques for genetic modification by transformation, mutagenesis or gene editing are well known to those skilled in the art and are described for example in “Strategies used for genetically modifying bacterial genome: site-directed mutagenesis, gene inactivation”, Journal of Zhejiang Univ-Sci B (Biomed & Biotechnol) 2016 17(2):83-99., and in Martinez-Garcia and de Lorenzo, “Pseudomonas putida in the quest of programmable chemistry”, Current Opinion in Biotechnology, 59:111-121, 2019.

Use will preferably be made, for integrating the mutated or recombinant genes in Pseudomonas putida, of the mutagenesis technique described in example 2.

In particular, a genetically modified strain may comprise a nucleic acid that modifies the expression of one or more genes that are naturally expressed in Pseudomonas putida.

According to a particular embodiment, a genetically modified strain may comprise a nucleic acid encoding one or more enzymes that are not naturally expressed in Pseudomonas putida.

The wild-type strains of P. putida KT2440 are available for example in the NBRC strain collection (National Institute of Technology and Evaluation Biological Resource center https://www.nite.go.jp/en/nbrc/, NBRC100650).

Furthermore, strains of Pseudomonas putida or Pseudomonas taiwanensis optimized for tyrosine production are known to those skilled in the art, who may use them as a founder strain to obtain the genetically modified strains according to the invention (Calero et al., 2016; Wierckx et al., 2005, Appl Environ Microbiol. 71(12):8221-7; Wynands et al., 2018; Otto et al. 2019, Front Bioeng Biotechnol November 20; 7: 312).

For the purposes of the present invention, the percentage identity refers to the percentage of identical residues in a nucleotide or amino acid sequence over a given fragment following alignment and comparison with a reference sequence. An alignment algorithm is used for the comparison and the sequences to be compared are input with the corresponding parameters of the algorithm. The default algorithm parameters can be used.

Preferably, for a nucleic acid sequence comparison and to determine a percentage identity, the BLAST algorithm, as described at https://blast.ncbi.nlm.nih.gov/Blast.cgi, is used with the default parameters.

For the purposes of the present invention, “mutated AroF1 gene” means a nucleic acid comprising at least a portion encoding a mutated version of 3-deoxy-D-arabino-heptulosonate-7-phosphate (DAHP) synthase under the control of a promoter enabling the expression thereof in the genetically modified strain.

For the purposes of the present invention, 3-deoxy-D-arabino-heptulosonate-7-phosphate (DAHP) synthase means the enzymes (EC 2.5.1.54) which are able, in bacteria, to carry out the first reaction in the shikimate pathway, which consists of the condensation of a phospho(enol)pyruvate (PEP) and an erthythrose-4-phosphate (E4P) to give DAHP.

According to a particular embodiment, the genetically modified strain of Pseudomonas putida comprises a mutated AroF-I gene encoding DAHP synthase, the amino acid sequence of which has at least 80%, 85%, 90%, 95% and most particularly at least 98% identity to the sequence SEQ ID NO:1, and having at least one P160L mutation, a P160L/Q164A double mutation, or a P160L/S193A double mutation, preferably a P160L/S193A double mutation.

The endogenous Pseudomonas putida AroF-I gene encodes DAHP synthase of amino acid sequence SEQ ID NO:1. In a particular embodiment, therefore, the genetically modified strain according to the present invention may comprise, in addition to the endogenous AroF-I gene, at least one recombinant mutated AroF-I nucleic acid sequence encoding a mutated protein comprising the P160L mutation, the P160L/Q164A double mutation, or the P160L/S193A double mutation, preferably the P160L/S193A double mutation.

According to a particular embodiment, the genetically modified strain of Pseudomonas putida comprises a mutated AroF-I gene encoding DAHP synthase comprising a P160L mutation as defined by amino acid sequence SEQ ID NO:2.

According to a variant of this embodiment, the modified strain of Pseudomonas putida comprises a mutated AroF-I gene encoding DAHP synthase, the sequence of which has at least 80%, 85%, 90%, 95% and most particularly at least 98% identity to the sequence SEQ ID NO: 2 and contains the P160L mutation.

According to another particular embodiment, the modified strain of Pseudomonas putida comprises a mutated AroF-I gene encoding DAHP synthase and having at least one P160L/Q164A double mutation. According to this embodiment, the mutated AroF-I gene encodes DAHP synthase comprising the P160L/Q164A double mutation defined by amino acid sequence SEQ ID NO: 3.

According to a variant of this embodiment, the modified strain of Pseudomonas putida comprises a mutated AroF-I gene encoding DAHP synthase, the sequence of which has at least 80%, 85%, 90%, 95% and most particularly at least 98% identity to the sequence SEQ ID NO: 3 and contains the P160L/Q164A double mutation.

According to a preferred embodiment, the modified strain of Pseudomonas putida comprises a mutated AroF-I gene encoding DAHP synthase, having at least one P160L/S193A double mutation. According to this embodiment, the mutated AroF-I gene encodes DAHP synthase comprising the P160L/S193A double mutation defined by amino acid sequence SEQ ID NO: 4.

According to a variant of this embodiment, the modified strain of Pseudomonas putida comprises a mutated AroF-I gene encoding DAHP synthase, the sequence of which has at least 80%, 85%, 90%, 95% and most particularly at least 98% identity to the sequence SEQ ID NO: 4 and contains the P160L/S193A double mutation.

In one embodiment, which may be combined with the preceding embodiments, the coding sequence of the mutated AroF-I gene is placed under the control of a heterologous promoter, in particular a constitutive or inducible promoter, for example selected from the promoters ptrc, xyls/pm or araC/pBAD, which makes it possible to overexpress the mutated AroF-I gene in the genetically modified strain according to the invention. In a preferred embodiment, the coding sequence of the mutated AroF-I gene in the genetically modified strain according to the invention is inserted so as to make the AroH gene non-functional, for example by disrupting the AroH gene or by deleting the AroH gene and in particular all or part of the coding sequence thereof.

The AroH gene encodes another DAHP synthase (isoenzyme of AroF). Thus, in one embodiment, the genetically modified strain according to the present invention comprises the deleted or disrupted AroH gene and at least one recombinant nucleic acid comprising the mutated AroF-I gene as described previously or a coding sequence of the mutated AroF-I gene.

In an entirely advantageous way, by implementing specific mutations the Applicant has developed a strain of Pseudomonas putida that is able to express a recombinant DAHP synthase which is resistant to feedback inhibition by tyrosine, thereby deregulating the tyrosine production pathway. In other words, the present invention makes it possible to produce strains that overproduce tyrosine as end product or as intermediate product in the synthesis of phenylpropanoid compounds. In particular, this overproduction is particularly advantageous for the production of phenylpropanoid compounds by the modified strains according to the invention.

Phenylpropanoid compounds are a class of plant-derived organic compounds that are biosynthesized from phenylalanine or tyrosine. As examples of phenylpropanoid compound, mention may particularly be made of coumaric acid, p-coumaroyl-CoA, 4-hydroxybenzalacetone, frambinone, zingerone, vanillin, flavonoids and stilbenoids.

For the purposes of the present invention, “strain that overproduces tyrosine” means a modified strain of Pseudomonas putida that is able to produce a greater quantity of tyrosine than a wild-type strain of Pseudomonas putida comprising the AroF-I gene encoding DAHP synthase of amino acid sequence SEQ ID NO:1 (non-mutated gene), either as end product or as intermediate product.

In order to be able to convert the tyrosine into phenylpropanoid compounds such as coumaric acid, the modified strain according to the invention may advantageously further comprise at least one additional recombinant gene.

For the purposes of the present invention, the expression “additional recombinant gene” means any recombinant gene present in the Pseudomonas putida strain in addition to the mutated AroF-I gene as defined previously. The additional recombinant gene may result from the insertion of a heterologous promoter, for example a strong promoter to overexpress an endogenous Pseudomonas putida gene, or a recombinant coding sequence encoding a protein that is not naturally expressed in Pseudomonas putida.

According to a particular embodiment, the genetically modified strain of Pseudomonas putida comprises at least an additional recombinant gene encoding a polypeptide having phenylalanine hydroxylase (phhA) activity and an additional recombinant gene encoding a polypeptide having tetrahydrobiopterin dehydratase (phhB) activity.

The Applicant observed that it was possible for these two enzymes, although present endogenously in Pseudomonasputida, to not be sufficiently active, and/or for their expression to be insufficient in the context of use of the strain for the production of phenylpropanoid compounds. According to this embodiment, activities have therefore been optimized by overexpressing these two enzymes, phhA and phhB. Overexpression may for example be obtained by placing the additional recombinant genes for these enzymes under the control of a heterologous promoter, in particular a constitutive or inducible promoter. Examples of such promoters are in particular the promoters ptrc, xyIS/pm, araC/pBAD.

The overexpression of the enzymes phhA and phhB is preferably obtained by placing the additional recombinant genes for these enzymes under the control of a strong inducible promoter araC/pBADopt (Prior et al., 2010).

The overexpression of a gene means greater expression of said gene in a genetically modified strain than in the same strain but in which the gene is expressed solely under the control of the natural promoter. Overexpression can be obtained by inserting one or more copies of the gene directly into the genome of the strain, preferably under the control of a strong promoter, or also by cloning in plasmids, in particular multicopy plasmids, preferably also under the control of a strong promoter.

In another embodiment, which can be combined with the preceding embodiment, the endogenous phhA and phhB coding sequences are placed under the control of a heterologous promoter as defined previously, for example in order to overexpress the corresponding endogenous coding sequences in the genetically modified strain according to the invention.

In particular, the modified strain may comprise an additional recombinant gene encoding a phenylalanine hydroxylase (phhA) (EC 1.14.16.1), the sequence of which is defined by the amino acid sequence SEQ ID NO: 5 or by a sequence having at least 80%, 85%, 90%, 95% and most particularly at least 98% identity to the sequence SEQ ID NO: 5 and encoding an enzyme having phhA activity, and an additional recombinant gene encoding a tetrahydrobiopterin dehydratase (phhB) (EC 4.2.1.96), the sequence of which is defined by the amino acid sequence SEQ ID NO: 6 or by a sequence having at least 80%, 85%, 90%, 95% and most particularly at least 98% identity to the sequence SEQ ID NO: 6 and encoding an enzyme having phhB activity, in particular under the control of a heterologous promoter enabling their overexpression.

According to this embodiment, the genetically modified strain according to the invention is able to overproduce tyrosine and also to convert phenylalanine into tyrosine via the phhA and phhB enzymes.

According to a particular embodiment, the additional recombinant genes encoding phenylalanine hydroxylase (phhA) and tetrahydrobiopterin dehydratase (phhB) comprise the corresponding coding sequences of Pseudomonas fluorescens (phhA VVN86558.1/phhB: AYF50180.1) or Pseudomonas aeruginosa (phhA AAA25936.1/phhB AAA25937.1).

According to a preferred embodiment, the modified strain of Pseudomonas putida comprises a mutated AroF-I gene encoding DAHP synthase comprising the P160L/S193A double mutation defined by the amino acid sequence SEQ ID NO: 4 or a sequence having at least, 85%, 90%, 95% and most particularly at least 98% identity to the sequence SEQ ID NO: 4, and the two additional recombinant genes below:
a recombinant gene encoding a phenylalanine hydroxylase (phhA), preferably a phhA defined by the sequence SEQ ID NO: 5 or a sequence having at least, 85%, 90%, 95% and most particularly at least 98% identity to the sequence SEQ ID NO: 5, and
a recombinant gene encoding a tetrahydrobiopterin dehydratase (phhB), preferably a phhB defined by the sequence SEQ ID NO: 6 or a sequence having at least, 85%, 90%, 95% and most particularly at least 98% identity to the sequence SEQ ID NO: 6;
said genes phhA and phhB preferably being placed under the control of a heterologous promoter, enabling their overexpression.

According to another particular embodiment, which may preferably be combined with the preceding embodiment, the genetically modified strain of Pseudomonas putida comprises an additional recombinant gene encoding a polypeptide having tyrosine ammonia lyase (TAL) activity. A recombinant gene encoding TAL may originate from the microorganism Rhodotorula glutinis and optimized according to the reference Zhou et al., 2015 (three point mutations in this TAL enzyme makes it more effective: S9N; A11T; E518V). This TAL enzyme is referred to as TAL_rg_opt. In particular, the modified strain may comprise a recombinant gene encoding a tyrosine ammonia lyase (TAL) (EC 4.3.1.23), the sequence of which is defined by the amino acid sequence SEQ ID NO: 7 (TAL_rg_opt) or by a sequence having at least 80%, 85%, 90%, 95% and most particularly at least 98% identity to the sequence SEQ ID NO: 7 and encoding an enzyme having TAL activity.

According to this particular embodiment, the genetically modified strain according to the invention is able to overproduce tyrosine and also to convert it into coumaric acid via the TAL enzyme.

According to another particular embodiment, which may be combined with the preceding embodiments, the genetically modified strain of Pseudomonas putida comprises an additional recombinant gene encoding 4-coumarate-CoA ligase (4-CL). In particular, the modified strain may comprise a recombinant gene encoding a 4-CL (EC 6.2.1.12), the sequence of which is defined by the amino acid sequence SEQ ID NO: 8 or by a sequence having at least 80%, 85%, 90%, 95% and most particularly at least 98% identity to the sequence SEQ ID NO: 8 and encoding an enzyme having 4-CL activity.

According to this particular embodiment, the genetically modified strain is able to convert coumaric acid into p-coumaroyl-CoA via the 4-CL enzyme.

According to another particular embodiment, which may preferably be combined with the preceding embodiments, the genetically modified strain of Pseudomonas putida comprises an additional recombinant gene encoding a polypeptide having benzalacetone synthase (BAS) activity. In particular, the modified strain may comprise a recombinant gene encoding a BAS (EC 2.3.1.212), the sequence of which is defined by the amino acid sequence SEQ ID NO: 9 or by a sequence having at least 80%, 85%, 90%, 95% and most particularly at least 98% identity to the sequence SEQ ID NO: 9 and encoding an enzyme having BAS activity.

According to this particular embodiment, the genetically modified strain is able to convert p-coumaroyl-CoA into 4-hydroxybenzalcetone via the BAS enzyme.

According to another particular embodiment, the genetically modified strain of Pseudomonas putida comprises a plurality of additional recombinant genes, namely the five additional recombinant genes below:
a recombinant gene encoding a phenylalanine hydroxylase (phhA), preferably a phhA defined by the sequence SEQ ID NO: 5, in particular under the control of a heterologous promoter enabling its overexpression,
a recombinant gene encoding a tetrahydrobiopterin dehydratase (phhB), preferably a phhB defined by the sequence SEQ ID NO: 6, in particular under the control of a heterologous promoter enabling its overexpression,
a recombinant gene encoding a tyrosine ammonia lyase (TAL_RG_OPT), preferably a TAL_RG_OPT defined by the sequence SEQ ID NO: 7,
a recombinant gene encoding a 4-coumarate-CoA ligase (4-CL), preferably a 4-CL defined by the sequence SEQ ID NO:8,
a recombinant gene encoding a benzalacetone synthase (BAS), preferably a BAS defined by the sequence SEQ ID NO:9.

The enzymes TAL, 4-CL and BAS are all enzymes involved in the synthesis of phenylpropanoid compounds.

According to this preferred embodiment, the modified strain is able to produce a multitude of phenylpropanoid compounds, namely in particular coumaric acid, p-coumaroyl-CoA and 4-hydroxybenzalacetone.

Another subject of the present invention relates to a genetically modified strain of Pseudomonas putida comprising an additional recombinant AroF-I gene encoding 3-deoxy-D-arabino-heptulosonate-7-phosphate (DAHP) synthase, the sequence of which has at least 80% identity to the sequence SEQ ID NO:1, and in which the genes encoding the enzymes phhA and phhB are overexpressed.

### Method for Synthesizing a Phenylpropanoid Compound

Another subject of the invention relates to a method for synthesizing one or more phenylpropanoid compounds.

The phenylpropanoid compounds may be as defined previously.

The synthesis method according to the invention comprises the implementation of a step of growing a genetically modified strain of Pseudomonas putida as defined previously in a culture medium under conditions that enable the expression of the mutated gene and/or of the additional recombinant genes required for the synthesis of one or more phenylpropanoid compounds.

According to a particular embodiment, the synthesis method according to the invention makes it possible to produce large quantities of coumaric acid.

According to a variant of this embodiment, the method may comprise a step of growing a genetically modified strain of Pseudomonas putida comprising a mutated AroF-I gene encoding 3-deoxy-D-arabino-heptulosonate-7-phosphate (DAHP) synthase, for example of sequence SEQ ID NO:2, SEQ ID NO:3 or SEQ ID NO:4, and at least the additional recombinant genes below, encoding:
a phenylalanine hydroxylase (phhA) of sequence SEQ ID NO: 5,
a tetrahydrobiopterin dehydratase (phhB) of sequence SEQ ID NO: 6,
and a tyrosine ammonia lyase (TAL_RG_OPT) of sequence SEQ ID NO: 7.

According to another particular embodiment, the synthesis method according to the invention makes it possible to produce frambinone, in particular industrial quantities thereof, and particularly with a yield at least equal to 20 g/l of frambinone in a fermenter of at least 500 I.

According to this embodiment, the method comprises a step of growing a genetically modified strain of Pseudomonas putida comprising a mutated AroF-I gene encoding 3-deoxy-D-arabino-heptulosonate-7-phosphate (DAHP) synthase of sequence SEQ ID NO:2, SEQ ID NO:3 or SEQ ID NO:4, and comprising the additional recombinant genes below, encoding:
a tyrosine ammonia lyase (TAL_RG_OPT), preferably a TAL_RG_OPT defined by the sequence SEQ ID NO: 7,
a 4-coumarate-CoA ligase (4-CL), preferably a 4-CL defined by the sequence SEQ ID NO:8,
a benzalacetone synthase (BAS), preferably a BAS defined by the sequence SEQ ID NO:9.

The synthesis method according to the invention may also comprise a step of purifying and/or recovering the phenylpropanoid compound such as coumaric acid or frambinone.

According to a particular embodiment, the strain is a genetically modified strain of Pseudomonas putida comprising an additional recombinant AroF-I gene encoding 3-deoxy-D-arabino-heptulosonate-7-phosphate (DAHP) synthase, the sequence of which has at least 80% identity to the sequence SEQ ID NO:1, and in which the genes encoding the enzymes phhA and phhB are overexpressed.

### Uses of the Strains According to the Invention

Another subject of the invention relates to the use of a strain of Pseudomonas putida as defined previously for the synthesis of phenylpropanoid compounds.

According to a preferred embodiment, the phenylpropanoid compound is selected from coumaric acid, p-coumaroyl-CoA, 4-hydroxybenzalacetone and frambinone, preferably from coumaric acid and/or frambinone.

The present invention will be better understood in light of the following nonlimiting examples, given solely by way of illustration and not intended to limit the scope of this invention as defined by the claims.

### EXAMPLES

### Example 1: Development of Mutants Enabling the Production of 3-Deoxy-D-Arabino-Heptulosonate-7-Phosphate (DAHP) Resistant to Tyrosine Feedback Inhibition

### A. In Silico Study

The starting enzyme used is the AroF-I enzyme identified as 3-deoxy-D-arabino-heptulosonate-7-phosphate (DAHP) in P. putida (Uniprot identifier Q88KG6).

The tertiary structure (in PDB format) of this enzyme was reconstructed from the protein sequence thereof, using the methodology described in Waterhouse, A. et al., (SWISS-MODEL: homology modeling of protein structures and complexes. Nucleic Acids Res. 46(W1), W296-W303 (2018)). The basic structure selected by sequence homology was that of the 3-deoxy-D-arabino-heptulosonate-7-phosphate synthase of Saccharomyces cerevisiae.

The region characterizing tyrosine feedback inhibition has been identified in the literature via a homologous protein in E. coli. Using an alignment of the tertiary structures of these two enzymes, the region corresponding to this feedback inhibition in AroF-I was identified.

Next, the docking of a tyrosine molecule with the AroF-I enzyme was carried out using the methodology described by O. Trott et al., (“AutoDock Vina: improving the speed and accuracy of docking with a new scoring function, efficient optimization and multithreading”, Journal of Computational Chemistry, 2010).

This method made it possible to identify the amino acids involved in the formation of chemical bonds with the phenylalanine in the docking pocket; namely, inter alia, the amino acids at positions 160 (P160), 164 (Q164), 190 (S190), 191 (G191), 193 (S193) and 225 (1225).

By searching in the literature and also in the NCBI and Uniprot databases, we identified protein sequences similar to AroF-I but having a certain amount of amino acid diversity, particularly at the positions corresponding to those involved in docking.

This approach made it possible to identify amino acids that can be substituted for the amino acids naturally found in the docking pocket of the AroF-I protein without modifying the structure or activity thereof.

Thus, only binding to tyrosine is inhibited by the mutations shown. Table 1 below lists the positions and original amino acids as well as the substitutes which characterize the mutations that could potentially deactivate the AroF-I feedback inhibition function.

TABLE 1

| Original amino acid | | |
|---|---|---|
| (AroF-I) | Position in FASTA | Substitute amino acids |
| P | 160 | A, L, T, V, M |
| Q | 164 | A, R |
| S | 190 | A, F |
| G | 191 | K |
| S | 193 | A, G |
| I | 225 | P, V, T |

Of the various mutations identified in table 1, a subset of mutations was chosen according to the properties of the substitute amino acids and also according to the impact of these mutations in evolutionarily-close proteins already described in the literature ((Cui, D et al., Molecular basis for feedback inhibition of tyrosine-regulated 3-deoxy-d-arabino-heptulosonate-7-phosphate synthase from Escherichia coli, 2019), (Ding, R. et al. Introduction of two mutations into AroG increases phenylalanine production in Escherichia coli. Biotechnol Lett 36, 2103-2108 (2014)), (Kikuchi Y et al. Mutational analysis of the feedback sites of phenylalanine-sensitive 3-deoxy-d-arabino-heptulosonate-7-phosphate synthase of Escherichia coli. Appl Environ Microbiol 63:761-762 (1997)).

The subset chosen is as follows: P160L, Q164A, S190A, G191K, S193A, and 1225P.

### B. Experiments

### Materials and Methods

### Cloning of Genes Encoding the AroF-I Enzyme and the Derived Mutants

In order to express and purify the AroF-I enzyme and the mutants derived therefrom, the corresponding genes are cloned in a pET28_b(+) plasmid with a His tag grafted to the N-terminal part of the protein (upstream of the gene's ATG).

To produce these constructs, use is made of the Gibson Assembly method (Gibson et al., 2009). The reaction product is transformed in E. coli BL21(DE3) strains (Novagen). The transformed cells are plated out on agar medium with an antibiotic for selection. The colonies obtained are subsequently verified using PCR and sequenced in order to validate them.

### Expression and Purification of the Enzymes

The E. coli BL21(DE3) strains containing the plasmids are cultured for 24 h in a ZYM autoinduction medium having the following composition:

a) Saline base:
yeast extract: 5 g/l
tryptone: 10 g/l
Na₂SO₄: 0.7 g/l
NH₄Cl: 2.7 g/l
KH₂PO₄: 3.4 g/l
Na₂HPO₄: 4.45 g/l

b) Solution of sugars (concentrated 50-fold):
Glycerol: 250 g/l
glucose: 25 g/l
lactose: 100 g/l

c) Solution of metals (concentrated 5000-fold):

TABLE 2

| | | | | Final | |
|---|---|---|---|---|---|
| | Molar | Concen- | Concen- | concen- | Per |
| Solution of | mass | tration | tration | tration | 100 |
| metals | (g/mol) | (g/l) | (mM) | (mM) | ml(g) |
| FeCl₃ | 162.2 | 40.55 | 250 | 0.050 | 4.05 |
| CaCl₂•2H₂O | 147.01 | 14.70 | 100 | 0.100 | 1.47 |
| MnCl₂ | 125.84 | 6.29 | 50 | 0.050 | 0.63 |
| ZnSO₄•7H₂O | 287.56 | 14.38 | 50 | 0.050 | 1.44 |
| CoCl₂ | 129.84 | 1.30 | 10 | 0.010 | 0.13 |
| CuCl₂ | 134.45 | 1.35 | 10 | 0.010 | 0.13 |
| NiCl₂ | 129.60 | 1.30 | 10 | 0.010 | 0.13 |
| Na₂MoO₄•2H₂O | 241.95 | 2.42 | 10 | 0.010 | 0.24 |
| Na₂SeO₃ | 172.94 | 1.73 | 10 | 0.010 | 0.17 |
| H3BO₃ | 61.83 | 0.62 | 10 | 0.010 | 0.06 |

d) Preparation of complete medium:

TABLE 3

| ZYM-5052 | Per 50 ml |
|---|---|
| ZYM saline base (ml) | 49.5 |
| 1M MgSO₄ (in μl) | 100 |
| Solution of metals, 5000-fold (in μl) | 10 |
| 5052 solution, 50-fold (in μl) | 1 |
| Antibiotic 50 mg/ml (in μl) | 50 |

The medium is seeded at OD=0.05 from an overnight preculture. The culture is carried out in a final volume of 50 ml in a 250 ml Erlenmeyer flask. The culture parameters are as follows: agitation at 140 rpm, 5 hours at 25° C. then 19 hours at 15° C.

All the chemical products used to prepare the medium come from Sigma-Aldrich, Roth or Euromedex.

After incubation, the 50 ml of culture are centrifuged for 10 minutes at 5000×g, 4° C. The supernatant is discarded and the pellet is taken up in 3 ml of LEW buffer (Macherey-Nagel, see below), 1 mg/ml of lyzosyme are added and the suspension is left over ice for 30 minutes.

The cells are subsequently lyzed using sonication (Omni Sonic Ruptor 400, Omni International, power 30%, pulse 40 for 8 minutes). After adding 0.22% of streptomycin, the samples are subsequently centrifuged for 30 minutes at 15 000×g, 4° C. The supernatant is recovered and the soluble proteins are purified following the supplier's recommendations, using the Protino® Ni-TED 2000 kit (Macherey-Nagel).

Finally, the purified proteins are concentrated ˜10-fold using Amicon Ultra-4 30Kd centrifugal filters (Merck), and the elution buffer is replaced with 0.1 M Tris-HCl, pH 7.5+10% glycerol. The purified proteins are stored at −80° C.

### Enzymatic Activity Assay

The activity of the enzymes is measured by monitoring the disappearance of the substrate using HPLC (High Pressure Liquid Chromatography) detection.

The reaction is performed in a final volume of 200 μl with 40 mM of phosphate buffer, pH 7, 300 μM of phospho(enol)pyruvate (PEP), 20 μg of purified enzyme, 1 mM of tyrosine (or another aromatic amino acid) or 3 μM of HCl, the volume being completed with ultrapure water.

After pre-incubation for 2 minutes at 30° C., the reaction is initiated by adding 300 μM of erythrose-4-phosphate (E4P) and the reaction mixture is incubated for 60 minutes at 30° C.

Finally, the mixture is heated for 5 minutes at 80° C. in order to stop the reaction and precipitate the proteins.

### Measurement of the Consumption of PEP Using HPLC

The enzymatic activity assays are firstly centrifuged for 10 minutes at 15 000×g in order to precipitate the proteins. The supernatant obtained is filtered over a 0.22 μm membrane before HPLC analysis.

The method used is described below:
System: Agilent 1100 (Agilent)
Column: Luna OMEGA polar C18 (Phenomenex)
Mobile phase: 1 mM phosphoric acid
Flow rate: 0.3 ml/min
UV detection: 220 nm
Injection: 5 μl
Duration of analysis: 15 minutes

In order to quantify the amount of PEP present in the samples, a series of standards is analyzed, with PEP concentrations from 0.1 mM to 1 mM.

### Measurement of the Concentration of PCA and CA Using HPLC

The column used is as follows: Kinetex® 5 μm F5 100A 150×4.6 mm
Mobile phases: 0.1% formic acid and acetonitrile
Gradient:
5 min: 100% formic acid
5 min to 25 min: 75% formic acid-25% acetonitrile
25 min to 30 min: 62% formic acid-38% acetonitrile
30 min to 35 min: 100% formic acid
Flow rate: 1 ml/min
Oven temperature: 40° C.
Sample injection: 10 μl
Coumaric acid detection: 315 nm
Cinnamic acid detection: 280 nm
   Cloning the phhaA/B Genes in an Arabinose-Dependent Expression Plasmid

The phhA/B genes are amplified by PCR directly from gDNA of the Pseudomonas putida strain KT2440 and are cloned in a pBBR1-MCS2 plasmid under the control of the araC/pBAD promoter.

To produce these constructs, use is made of the Gibson Assembly method (Gibson et al., 2009). The reaction product is transformed in the E. coli BL21(DE3) strain (Novagen). The transformed cells are plated out on agar medium with an antibiotic for selection. The colonies obtained are subsequently verified using PCR and the plasmids are sequenced in order to validate them.

The expression plasmid is subsequently transformed in the donor E. coli strain S17.1 in order to be transferred into the Pseudomonas putida strains of interest by conjugation.

### Results

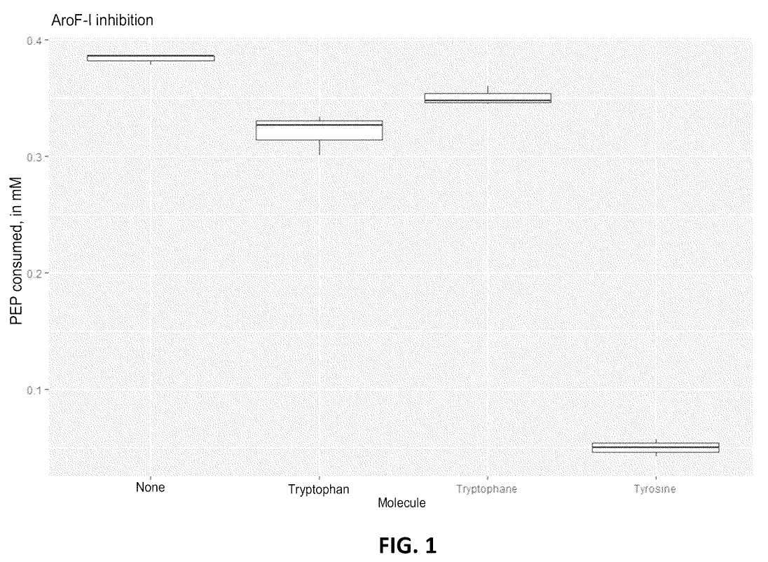

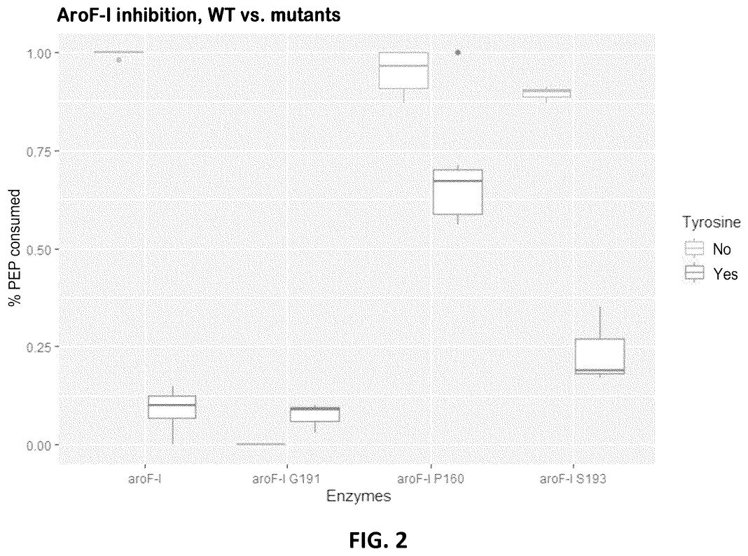

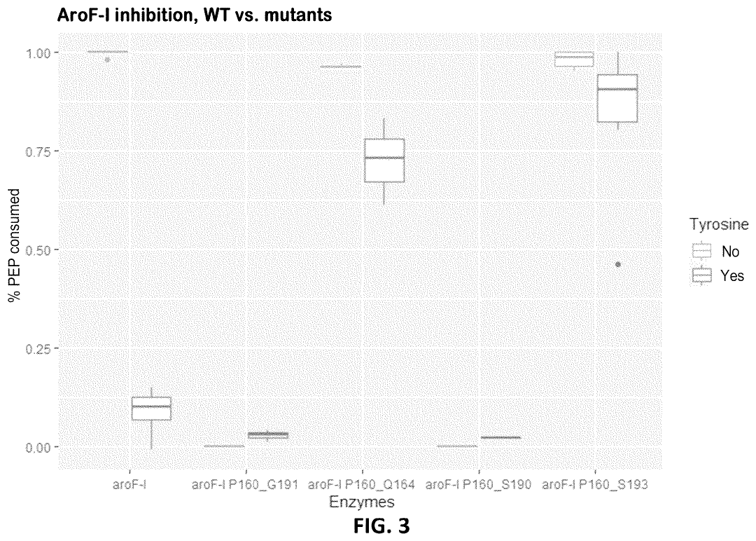

The activity of the wild-type Pseudomonas putida AroF-I enzyme is indeed inhibited by tyrosine (FIG. 1). Introduction of the P160L mutation makes it possible to make the enzyme partially resistant to this inhibition by tyrosine (FIG. 2), and the effect can be slightly improved by introducing a P160L/Q164A double mutation (FIG. 3). However, the P160L/G191K and P160L/S190 double mutations have a deleterious effect on the AroF-I enzyme, which no longer has any activity, whether in the presence or absence of tyrosine (FIG. 3).

In contrast, and particularly beneficially and surprisingly, the introduction of a P160L/S193A double mutation restored virtually all the enzymatic activity and makes the AroF-I enzyme virtually completely resistant to tyrosine inhibition (FIG. 3).

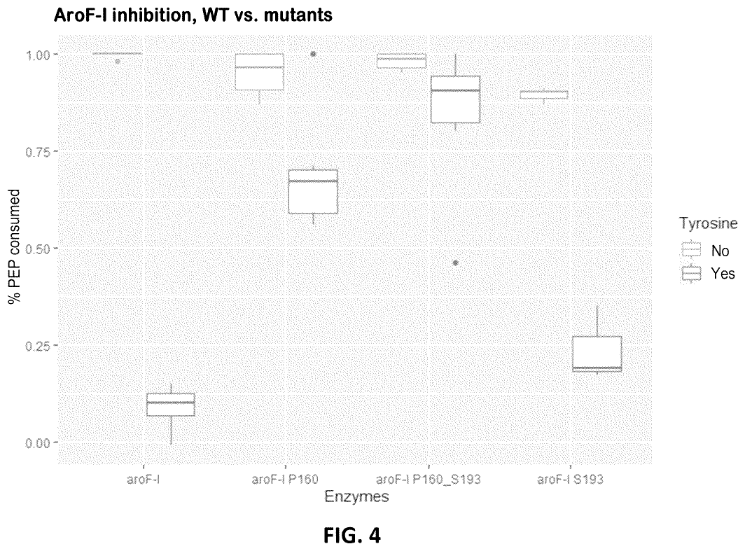

These results are confirmed by FIG. 4, which shows the synergistic effect of this double mutation on the feedback inhibition caused by tyrosine compared to a single P160L or S193A mutation.

### Example 2: Genetically Modified Strains of Pseudomonas putida for The Synthesis of Phenylpropanoid Compounds

In order to enable the biosynthesis of phenylpropanoid compounds, a plurality of genetic modifications are made within the Pseudomonas putida strain. To this end, additional recombinant genes encoding enzymes for the synthesis of phenylpropanoid compounds such as coumaric acid or frambinone are integrated in the Pseudomonas putida chromosome according to the following protocol.

Protocol for Genetic Mutagenesis of Pseudomonas putida:

Genetic mutagenesis in Pseudomonas putida is carried out by means of suicide plasmids which integrate into the chromosome and then leave it again, leaving behind the desired gene deletions or insertions. The suicide plasmid used is pK18mobsacB (Schäfer A, Tauch A, Jäger W, Kalinowski J, Thierbach G, Pühler A. Small mobilizable multi-purpose cloning vectors derived from the Escherichia coli plasmids pK18 and pK19: selection of defined deletions in the chromosome of Corynebacterium glutamicum. Gene 1994 Jul. 22; 145(1): 69-73). This plasmid carries a kanamycin antibiotic resistance cassette and the sacB counter-selection cassette. It also comprises an origin of replication which only works in the bacterium E. coli, and an origin of transfer, oriT, which enables it to be transferred by conjugation from E. coli to another bacterial strain such as Pseudomonas putida.

The genes to be inserted are cloned in this plasmid, as are the homologous regions at the chosen insertion site in the bacterial chromosome. These homologous regions are cloned on either side of the gene to be inserted, and should have a minimum size of 800 base pairs.

The clonings are carried out in an Escherichia coli strain that is able to conjugate (in general, the strain S17.1, Simon, R., Priefer, U. and A. Pulher, A broad host range mobilization system for in vivo genetic engineering: transposon mutagenesis in gram negative bacteria. Nature BioTechnology volume 1, pages 784-791 (1983)).

The conjugation protocol is as follows:

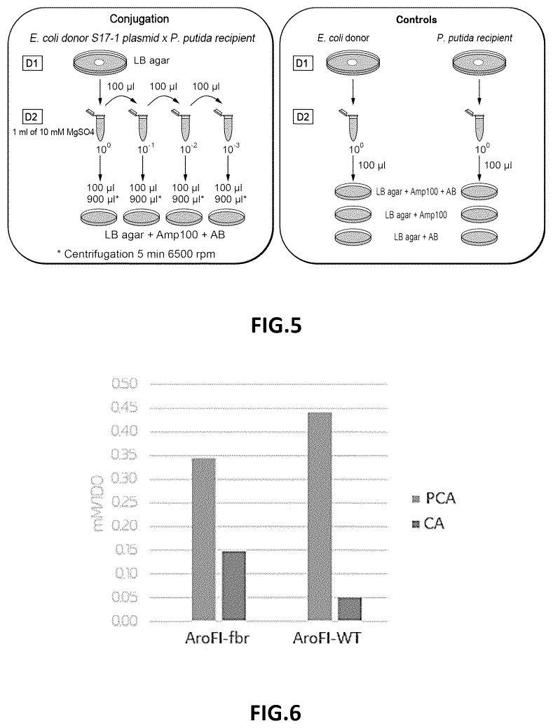

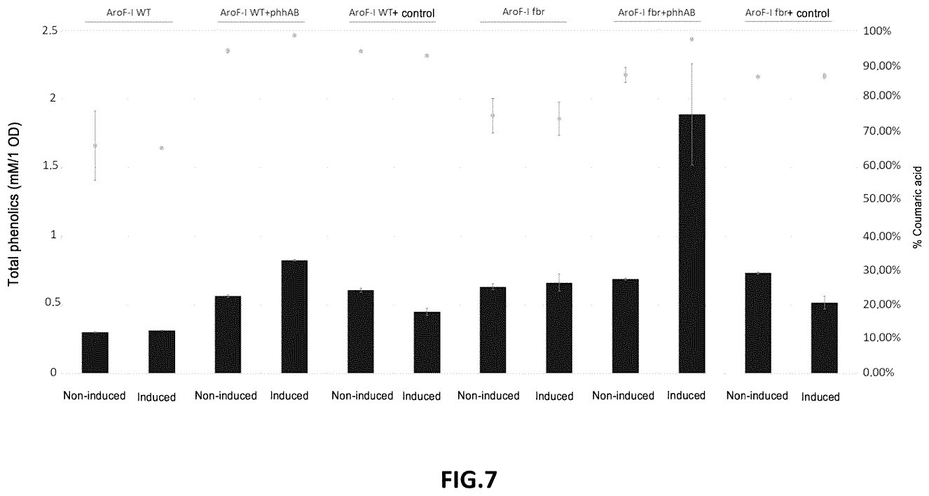

A culture droplet of S17.1/suicide plasma (donor strain) is deposited on an LB agar rich medium (Luria-Miller, Roth, reference X968.2, containing 1.5% agar), and a culture droplet of the Pseudomonas putida strain (recipient strain) is deposited on the first droplet. The culture dish is incubated overnight at 30° C. The droplet is subsequently diluted in 10 mM MgSO4 and the protocol is carried out as described in FIG. 6.

The pK18mobsacB conjugation plasmid is an integrative plasmid, able to integrate into the genome of the recipient bacterial strain in order to produce transconjugants. Because the plasmid is resistant to kanamycin, the selection medium for the transconjugants is LB agar containing 100 mg/ml ampicillin (Pseudomonas putida is naturally resistant to this antibiotic), and 50 mg/ml kanamycin (kanamycin, ROTH reference T832.4; ampicillin, EUROMEDEX, reference EUO400-D). This medium thus makes it possible to select the Pseudomonas putida in which a plasmid has been integrated.

The rest of the protocol comprises the following steps:
Excision of the suicide plasmid: sample 8 Kn+AmpR clones and culture them in 10 ml of 100 mg/ml LB+Amp medium (dilution 1/2000) at 30° C. with agitation. Incubate for between 12 h and 24 h.
Counter-selection sacB on 25% sucrose: streak the culture of the pool of clones on YT agar and YT+sucrose 25% agar. Incubate at 30° C. until the following morning. Re-sample 20-25 clones of sucrose+YT agar, YT+sucrose 25% agar, and YT agar+Kn 50 mg/ml. Incubate at 30° C. until the following morning. YT: Yeast extract tryptone
Verification of gene insertion using PCR.

Primers that hybridize on either side of the chromosomal insertion region make it possible to verify the mutant clones by carrying out colony PCR for the KnS and Sucrose+clones.

Genes to be Inserted into Pseudomonas putida

One or more of the genes listed below are inserted into Pseudomonas putida in order to enable the biosynthesis of phenylpropanoid compounds, for instance coumaric acid or frambinone.

These genes have been specifically identified and selected in known microorganisms. The genes are synthesized and the codons are optimized for maximum expression in Pseudomonas putida.

Some genes are endogenous to Pseudomonas putida, but it is recommended to test the activity of the proteins they code for and, if these enzymes are not active enough or are insufficiently expressed, these activities can be optimized.

### Genes Encoding a Mutated 3-Deoxy-D-Arabino-Heptulosonate-7-Phosphate (DAHP) Synthase Enzyme:

SEQ ID NO:10: sequence of the 3-deoxy-D-arabino-heptulosonate-7-phosphate (DAHP) synthase gene with P160L mutation (Pseudomonas putida KT2440), encoding DAHP synthase of sequence SEQ ID NO:2.

SEQ ID NO:11: sequence of the 3-deoxy-D-arabino-heptulosonate-7-phosphate (DAHP) synthase gene with P160L/Q164A double mutation (Pseudomonas putida KT2440), encoding DAHP synthase of sequence SEQ ID NO:3.

SEQ ID NO:12: sequence of the 3-deoxy-D-arabino-heptulosonate-7-phosphate (DAHP) synthase gene with P160L/S193A double mutation (Pseudomonas putida KT2440), encoding DAHP synthase of sequence SEQ ID NO:4.

### Additional Genes Enabling the Synthesis of Phenylpropanoid Compounds

SEQ ID NO:13: gene (Pseudomonas putida KT2440) for phenylalanine hydroxylase (phhA), encoding the phhA of sequence SEQ ID NO:5.

SEQ ID NO:14: gene (Pseudomonas putida KT2440) for tetrahydrobiopterin dehydratase (phhB), encoding the phhB of sequence SEQ ID NO:6.

SEQ ID NO:15: gene (Rhodotorula glutinis) for tyrosine ammonia lyase, encoding the beta-xylosidase of sequence SEQ ID NO:7.

SEQ ID NO:16: gene (Pseudomonas putida KT2440 fcs gene) for 4-coumarate-CoA ligase (4-CL), encoding the 4-CL of sequence SEQ ID NO:8.

SEQ ID NO:17: gene (Rheum palmatum) for benzalacetone synthase (BAS), encoding the BAS of sequence SEQ ID NO: 9.

### Example 3: In Vivo Activity of a Genetically Modified Strain of Pseudomonas putida According to the Invention

### Preparation of an AroF-I-Fbr P160L/S193A Mutant

A Pseudomonas putida KT2440 strain was genetically modified as described in example 1 so as to express the AroF-I gene encoding DAHP synthase comprising the P160L/S193A double mutation defined by the amino acid sequence SEQ ID NO: 4 and to express the additional recombinant gene encoding a heterologous tyrosine ammonia lyase (TAL_RG_OPT) of sequence SEQ ID NO: 7.

This strain is referred to as “AroF-I-fbr P160L/S193A mutant”.

The activity of the AroF-I-fbr P160L/S193A mutant on the production of coumaric acid (PCA) or cinnamic acid (CA) was determined by measuring the quantities produced of these acids compared to a Pseudomonas putida KT2440 strain genetically modified to express the wild-type AroF-I enzyme, the latter strain serving as control (AroF-I-WT mutant).

### Results

Cinnamic acid production increases significantly in a P. putida strain expressing the AroF-I enzyme having the P160L/S193A double mutation (FIG. 6).

### Optimization of the AroF-I-Fbr P160L/S193A Mutant for the Production Of Coumaric Acid.

The AroF-I-fbr P160L/S193A mutant was modified in order to express the additional recombinant genes encoding a phenylalanine hydroxylase (phhA) of sequence SEQ ID NO: 5, and a tetrahydrobiopterin dehydratase (phhB) of sequence SEQ ID NO: 6. The genes encoding these enzymes were cloned in a plasmid and expressed under the control of the araC/pBADopt inducible promoter, induction being carried out with 0.5% arabinose: plasmid pC2F387.

This strain is referred to as “optimized AroF-I-fbr mutant”.

The production of coumaric acid (PCA) or cinnamic acid (CA) by the optimized AroF-I-fbr mutant was measured then compared to that obtained with the AroF-I-fbr P160L/S193A mutant and that obtained with two control strains, namely:
a Pseudomonas putida KT2440 strain expressing the wild-type AroF-I enzyme (AroF-I-WT mutant), and
a Pseudomonas putida KT2440 strain expressing the wild-type AroF-I and the two phhA and phhB enzymes of sequences SEQ ID NO:5 and SEQ ID NO:6, respectively (AroF-I-WT phhA/B mutant).

### Results

The results presented in FIG. 6 show that the overexpression of the phhA and phhB genes in the AroF-I-WT mutant strain leads to greater production of phenolic acids (PCA+CA).

Particularly interestingly, the optimized AroF-I-fbr mutant makes it possible to obtain production of total phenolic acids that is much higher than the AroF-I-fbr P160L/S193A mutant and the AroF-I-WT phhA/B mutant. Proportionally, coumaric acid represents virtually all of the total phenolics produced, and this is advantageous since cinnamic acid is not an industrially useful compound for the production of phenylpropanoid compounds.

These results clearly demonstrate that the optimized AroF-I-fbr mutant is particularly suited to the production of phenylpropanoid compounds, and particularly of coumaric acid.

### Free Sequence Listing Text

In the present application, reference is made to sequence listings, the identifiers (“SEQ ID NO”) of which are listed in the table below. Regardless of the form in which the listings are provided, they form part of the present application.

TABLE 4

| | |
|---|---|
| SEQ ID NO: 1 | LAAGTRDLTSNTMADLPIDDLNVASNETLITPDQLKKE |
| AA | IPLSAKALQTVTAGREWVRNILDGKDHRLFVVVGPCSI |
| Wild-type | HDIKAAHEYAERLKVLAEEVSDTLYLVMRVYFEKPRTT |
| AroF-I | VGWKGLINDPYLDDSFKIQDGLHIGRKLLLDLAEMGLP |
| | TATEALDPISPQYLQDLISWSAIGARTTESQTHREMAS |
| | GLSSAVGFKNGTDGGLTVAINALQSVSKPHRFLGINQE |
| | GGVSIVTTKGNPYGHWVLRGGNGKPNYDSVSVALCEQD |
| | LAKAKIKANIMVDCSHANSNKDPALQPLVMENVANQIL |
| | EGNQSIIGLMVESHLNWGCQSIPKNLDDLQYGVSITDA |
| | CIDWSATEKTLRSMHAKLKDVLPQRKRG |
| SEQ ID NO: 2 | LAAGTRDLTSNTMADLPIDDLNVASNETLITPDQLKKE |
| AA | IPLSAKALQTVTAGREWVRNILDGKDHRLFVVVGPCSI |
| AroF-I_P160L | HDIKAAHEYAERLKVLAEEVSDTLYLVMRVYFEKPRTT |
| | VGWKGLINDPYLDDSFKIQDGLHIGRKLLLDLAEMGLP |
| | TATEALDLISPQYLQDLISWSAIGARTTESQTHREMAS |
| | GLSSAVGFKNGTDGGLTVAINALQSVSKPHRFLGINQE |
| | GGVSIVTTKGNPYGHVVLRGGNGKPNYDSVSVALCEQD |
| | LAKAKIKANIMVDCSHANSNKDPALQPLVMENVANQIL |
| | EGNQSIIGLMVESHLNWGCQSIPKNLDDLQYGVSITDA |
| | CIDWSATEKTLRSMHAKLKDVLPQRKRG |
| SEQ ID NO: 3 | LAAGTRDLTSNTMADLPIDDLNVASNETLITPDQLKKE |
| AA | IPLSAKALQTVTAGREWVRNILDGKDHRLFVVVGPCSI |
| AroF-I_P160L/ | HDIKAAHEYAERLKVLAEEVSDTLYLVMRVYFEKPRTT |
| Q164A | VGWKGLINDPYLDDSFKIQDGLHIGRKLLLDLAEMGLP |
| | TATEALDLISPAYLQDLISWSAIGARTTESQTHREMAS |
| | GLSSAVGFKNGTDGGLTVAINALQSVSKPHRFLGINQE |
| | GGVSIVTTKGNPYGHVVLRGGNGKPNYDSVSVALCEQD |
| | LAKAKIKANIMVDCSHANSNKDPALQPLVMENVANQIL |
| | EGNQSIIGLMVESHLNWGCQSIPKNLDDLQYGVSITDA |
| | CIDWSATEKTLRSMHAKLKDVLPQRKRG |
| SEQ ID NO: 4 | LAAGTRDLTSNTMADLPIDDLNVASNETLITPDQLKKE |
| AA | IPLSAKALQTVTAGREWVRNILDGKDHRLFVVVGPCSI |
| AroF-I_P160L/ | HDIKAAHEYAERLKVLAEEVSDTLYLVMRVYFEKPRTT |
| S193A | VGWKGLINDPYLDDSFKIQDGLHIGRKLLLDLAEMGLP |
| | TATEALDLISPQYLQDLISWSAIGARTTESQTHREMAS |
| | GLASAVGFKNGTDGGLTVAINALQSVSKPHRFLGINQE |
| | GGVSIVTTKGNPYGHVVLRGGNGKPNYDSVSVALCEQD |
| | LAKAKIKANIMVDCSHANSNKDPALQPLVMENVANQIL |
| | EGNQSIIGLMVESHLNWGCQSIPKNLDDLQYGVSITDA |
| | CIDWSATEKTLRSMHAKLKDVLPQRKRG |
| SEQ ID NO: 5 | MKQTQYVAREPDAHGFIDYPQQEHAVWNTLITRQLKVI |
| AA | EGRACQEYLDGIDQLKLPHDRIPQLGEINKVLGATTGW |
| Phenylalanine | QVARVPALIPFQTFFELLASKRFPVATFIRTPEELDYL |
| hydroxylase (phhA) | QEPDIFHEIFGHCPLLTNPWFAEFTHTYGKLGLAATKE |
| | QRVYLARLYWMTIEFGLMETAQGRKIYGGGILSSPKET |
| | VYSLSDEPEHQAFDPIEAMRTPYRIDILQPVYFVLPNM |
| | KRLFDLAHEDIMGMVHKAMQLGLHAPKFPPKVAA |
| SEQ ID NO: 6 | MNALNQAHCEACRADAPKVTDEELAELIREIPDWNIEV |
| AA | RDGHMELERVFLFKNFKHALAFTNAVGEIAEAEGHHPG |
| Tetrahydrobiopterin | LLTEWGKVTVTWWSHSIKGLHRNDFIMCARTDKVAETA |
| dehydratase (phhB) | EGRK |
| SEQ ID NO: 7 | MAPRPTSQNQTRTCPTTQVTQVDIVEKMLAAPTDSTLE |
| AA | LDGYSLNLGDVVSAARKGRPVRVKDSDEIRSKIDKSVE |
| Tyrosine ammonia | FLRSQLSMSVYGVTTGFGGSADTRTEDAISLQKALLEH |
| lyase | QLCGVLPSSFDSFRLGRGLENSLPLEVVRGAMTIRVNS |
| (TAL_RG_OPT) | LTRGHSAVRLVVLEALTNFLNHGITPIVPLRGTISASG |
| | DLSPLSYIAAAISGHPDSKVHVVHEGKEKILYAREAMA |
| | LFNLEPVVLGPKEGLGLVNGTAVSASMATLALHDAHML |
| | SLLSQSLTAMTVEAMVGHAGSFHPFLHDVTRPHPTQIE |
| | VAGNIRKLLEGSRFAVHHEEEVKVKDDEGILRQDRYPL |
| | RTSPQWLGPLVSDLIHAHAVLTIEAGQSTTDNPLIDVE |
| | NKTSHHGGNFQAAAVANTMEKTRLGLAQIGKLNFTQLT |
| | EMLNAGMNRGLPSCLAAEDPSLSYHCKGLDIAAAAYTS |
| | ELGHLANPVTTHVQPAEMANQAVNSLALISARRTTESN |
| | DVLSLLLATHLYCVLQAIDLRAIVFEFKKQFGPAIVSL |
| | IDQHFGSAMTGSNLRDELVEKVNKTLAKRLEQTNSYDL |
| | VPRWHDAFSFAAGTVVEVLSSTSLSLAAVNAWKVAAAE |
| | SAISLTRQVRETFWSAASTSSPALSYLSPRTQILYAFV |
| | REELGVKARRGDVFLGKQEVTIGSNVSKIYEAIKSGRI |
| | NNVLLKMLA |
| SEQ ID NO: 8 | MNNEARSGSTDPGQRPRYRQVAIGHPQVQVSHVDDVLR |
| AA | MQPVEPLAPLPARLLERLVHWAQVRPDTTFIAARQADG |
| 4-coumarate-CoA | AWRSISYVQMLADVRTIAANLLGLGLSAERPLALLSGN |
| ligase (4-CL) | DIEHLQIALGAMYAGIAYCPVSPAYALLSQDFAKLRHV |
| | CEVLTPGVVFVSDSQPFQRAFEAVLDDSVGVISVRGQV |
| | AGRPHISFDSLLQPGDLAAADAAFAATGPDTIAKFLFT |
| | SGSTKLPKAVITTQRMLCANQQMLLQTFPTFAEEPPVL |
| | VDWLPWNHTFGGSHNLGIVLYNGGSFYLDAGKPTPQGF |
| | AETLRNLREISPTAYLTVPKGWEELVKALEQDPALREV |
| | FFARIKLFFFAAAGLSQSVWDRLDRIAEQHCGERIRMM |
| | AGLGMTEASPSCTFTTGPLSMAGYVGLPAPGCEVKLVP |
| | VGDKLEARFRGPHIMPGYWRSPQQTAEAFDEEGFYCSG |
| | DALKLADARQPELGLMFDGRIAEDFKLSSGVFVSVGPL |
| | RNRAVLEGSPYVQDIVVTAPDRECLGLLVFPRLPECRR |
| | LAGLAEDASDARVLANDTVRSWFADWLERLNRDAQGNA |
| | SRIEWLSLLAEPPSIDAGEITDKGSINQRAVLQRRAAQ |
| | VEALYRGEDPDALHAKVRP |
| SEQ ID NO: 9 | MATEEMKKLATVMAIGTANPPNCYYQADFPDFYFRVTN |
| AA | SDHLINLKQKFKRLCENSRIEKRYLHVTEEILKENPNI |
| benzalacetone | AAYEATSLNVRHKMQVKGVAELGKEAALKAIKEWGQPK |
| synthase (BAS) | SKITHLIVCCLAGVDMPGADYQLTKLLDLDPSVKRFMF |
| | YHLGCYAGGTVLRLAKDIAENNKGARVLIVCSEMTTTC |
| | FRGPSETHLDSMIGQAILGDGAAAVIVGADPDLTVERP |
| | IFELVSTAQTIVPESHGAIEGHLLESGLSFHLYKTVPT |
| | LISNNIKTCLSDAFTPLNISDWNSLFWIAHPGGPAILD |
| | QVTAKVGLEKEKLKVTRQVLKDYGNMSSATVFFIMDEM |
| | RKKSLENGQATTGEGLEWGVLFGFGPGITVETWLRSVP |
| | VIS |
| SEQ ID NO: 10 | 5′-TTGGCGGCCGGCACCCGTGACCTGACGAGTAACAC |
| Nt | GATGGCTGATTTACCGATCGATGACTTGAACGTTGCCT |
| AroF-I_P160L | CCAACGAGACCCTGATCACCCCTGATCAGCTCAAGAAG |
| | GAAATCCCCCTCAGCGCCAAGGCCCTGCAGACCGTGAC |
| | TGCCGGCCGTGAAGTGGTGCGCAATATTCTCGACGGCA |
| | AGGACCATCGCCTGTTCGTCGTGGTCGGCCCTTGCTCC |
| | ATCCACGACATCAAGGCAGCCCACGAATACGCCGAGCG |
| | CCTGAAAGTGCTGGCCGAAGAAGTGTCCGATACGCTGT |
| | ACCTGGTCATGCGCGTGTACTTCGAAAAGCCGCGCACC |
| | ACCGTCGGCTGGAAAGGCCTGATCAACGATCCGTACCT |
| | GGATGACTCGTTCAAGATCCAGGACGGCCTGCACATCG |
| | GCCGCAAGTTGCTGCTGGACCTGGCCGAAATGGGCCTG |
| | CCGACCGCCACCGAAGCGCTCGACCTGATTTCGCCGCA |
| | GTACCTGCAAGACCTGATCAGCTGGTCGGCCATCGGTG |
| | CCCGCACCACCGAATCGCAAACACACCGCGAGATGGCC |
| | TCGGGCCTGTCCTCGGCGGTGGGTTTCAAGAACGGTAC |
| | CGATGGCGGCCTGACCGTTGCCATCAATGCCCTGCAGT |
| | CGGTGTCCAAGCCGCACCGCTTCCTGGGCATCAACCAG |
| | GAAGGCGGCGTGTCGATCGTCACCACCAAGGGCAACCC |
| | ATACGGCCACGTGGTACTGCGCGGCGGCAATGGCAAGC |
| | CGAACTACGACTCGGTCAGCGTCGCCCTGTGCGAACAG |
| | GACCTGGCCAAGGCCAAGATCAAGGCCAACATCATGGT |
| | CGACTGCAGCCATGCCAACTCCAACAAGGACCCGGCCC |
| | TGCAACCGCTGGTGATGGAGAACGTCGCCAACCAGATT |
| | CTCGAAGGCAACCAGTCGATCATCGGCCTGATGGTCGA |
| | AAGCCACCTGAACTGGGGCTGTCAGTCCATTCCGAAAA |
| | ACCTGGACGATTTGCAGTATGGCGTGTCGATCACGGAC |
| | GCCTGCATCGACTGGTCGGCTACCGAGAAAACCCTGCG |
| | CAGCATGCATGCCAAGCTCAAGGATGTGCTGCCGCAGC |
| | GTAAGCGCGGCTGA-3′ |
| SEQ ID NO: 11 | 5′-TTGGCGGCCGGCACCCGTGACCTGACGAGTAACAC |
| Nt | GATGGCTGATTTACCGATCGATGACTTGAACGTTGCCT |
| AroF-I_P16L/Q164A | CCAACGAGACCCTGATCACCCCTGATCAGCTCAAGAAG |
| | GAAATCCCCCTCAGCGCCAAGGCCCTGCAGACCGTGAC |
| | TGCCGGCCGTGAAGTGGTGCGCAATATTCTCGACGGCA |
| | AGGACCATCGCCTGTTCGTCGTGGTCGGCCCTTGCTCC |
| | ATCCACGACATCAAGGCAGCCCACGAATACGCCGAGCG |
| | CCTGAAAGTGCTGGCCGAAGAAGTGTCCGATACGCTGT |
| | ACCTGGTCATGCGCGTGTACTTCGAAAAGCCGCGCACC |
| | ACCGTCGGCTGGAAAGGCCTGATCAACGATCCGTACCT |
| | GGATGACTCGTTCAAGATCCAGGACGGCCTGCACATCG |
| | GCCGCAAGTTGCTGCTGGACCTGGCCGAAATGGGCCTG |
| | CCGACCGCCACCGAAGCGCTCGACCTGATTTCGCCGGC |
| | CTACCTGCAAGACCTGATCAGCTGGTCGGCCATCGGTG |
| | CCCGCACCACCGAATCGCAAACACACCGCGAGATGGCC |
| | TCGGGCCTGTCCTCGGCGGTGGGTTTCAAGAACGGTAC |
| | CGATGGCGGCCTGACCGTTGCCATCAATGCCCTGCAGT |
| | CGGTGTCCAAGCCGCACCGCTTCCTGGGCATCAACCAG |
| | GAAGGCGGCGTGTCGATCGTCACCACCAAGGGCAACCC |
| | ATACGGCCACGTGGTACTGCGCGGCGGCAATGGCAAGC |
| | CGAACTACGACTCGGTCAGCGTCGCCCTGTGCGAACAG |
| | GACCTGGCCAAGGCCAAGATCAAGGCCAACATCATGGT |
| | CGACTGCAGCCATGCCAACTCCAACAAGGACCCGGCCC |
| | TGCAACCGCTGGTGATGGAGAACGTCGCCAACCAGATT |
| | CTCGAAGGCAACCAGTCGATCATCGGCCTGATGGTCGA |
| | AAGCCACCTGAACTGGGGCTGTCAGTCCATTCCGAAAA |
| | ACCTGGACGATTTGCAGTATGGCGTGTCGATCACGGAC |
| | GCCTGCATCGACTGGTCGGCTACCGAGAAAACCCTGCG |
| | CAGCATGCATGCCAAGCTCAAGGATGTGCTGCCGCAGC |
| | GTAAGCGCGGCTGA-3′ |
| SEQ ID NO: 12 | 5′-TTGGCGGCCGGCACCCGTGACCTGACGAGTAACAC |
| Nt | GATGGCTGATTTACCGATCGATGACTTGAACGTTGCCT |
| AroF-I_P160L/ | CCAACGAGACCCTGATCACCCCTGATCAGCTCAAGAAG |
| S193A | GAAATCCCCCTCAGCGCCAAGGCCCTGCAGACCGTGAC |
| | TGCCGGCCGTGAAGTGGTGCGCAATATTCTCGACGGCA |
| | AGGACCATCGCCTGTTCGTCGTGGTCGGCCCTTGCTCC |
| | ATCCACGACATCAAGGCAGCCCACGAATACGCCGAGCG |
| | CCTGAAAGTGCTGGCCGAAGAAGTGTCCGATACGCTGT |
| | ACCTGGTCATGCGCGTGTACTTCGAAAAGCCGCGCACC |
| | ACCGTCGGCTGGAAAGGCCTGATCAACGATCCGTACCT |
| | GGATGACTCGTTCAAGATCCAGGACGGCCTGCACATCG |
| | GCCGCAAGTTGCTGCTGGACCTGGCCGAAATGGGCCTG |
| | CCGACCGCCACCGAAGCGCTCGACCTGATTTCGCCGCA |
| | GTACCTGCAAGACCTGATCAGCTGGTCGGCCATCGGTG |
| | CCCGCACCACCGAATCGCAAACACACCGCGAGATGGCC |
| | TCGGGTTTGGCATCGGCGGTGGGTTTCAAGAACGGTAC |
| | CGATGGCGGCCTGACCGTTGCCATCAATGCCCTGCAGT |
| | CGGTGTCCAAGCCGCACCGCTTCCTGGGCATCAACCAG |
| | GAAGGCGGCGTGTCGATCGTCACCACCAAGGGCAACCC |
| | ATACGGCCACGTGGTACTGCGCGGCGGCAATGGCAAGC |
| | CGAACTACGACTCGGTCAGCGTCGCCCTGTGCGAACAG |
| | GACCTGGCCAAGGCCAAGATCAAGGCCAACATCATGGT |
| | CGACTGCAGCCATGCCAACTCCAACAAGGACCCGGCCC |
| | TGCAACCGCTGGTGATGGAGAACGTCGCCAACCAGATT |
| | CTCGAAGGCAACCAGTCGATCATCGGCCTGATGGTCGA |
| | AAGCCACCTGAACTGGGGCTGTCAGTCCATTCCGAAAA |
| | ACCTGGACGATTTGCAGTATGGCGTGTCGATCACGGAC |
| | GCCTGCATCGACTGGTCGGCTACCGAGAAAACCCTGCG |
| | CAGCATGCATGCCAAGCTCAAGGATGTGCTGCCGCAGC |
| | GTAAGCGCGGCTGA-3′ |
| SEQ ID NO: 13 | 5′-ATGAAACAGACGCAATACGTGGCACGCGAGCCCGA |
| Nt | TGCGCATGGTTTTATCGATTACCCGCAGCAAGAGCATG |
| Phenylalanine | CGGTGTGGAACACCCTGATCACCCGCCAGCTGAAAGTG |
| hydroxylase (phhA) | ATCGAAGGCCGTGCGTGCCAGGAATACCTGGACGGCAT |
| | CGACCAGCTGAAATTGCCGCATGACCGCATTCCGCAAC |
| | TGGGCGAGATCAACAAGGTGCTGGGTGCCACCACCGGC |
| | TGGCAGGTTGCCCGGGTTCCGGCGCTGATCCCCTTCCA |
| | GACCTTCTTCGAATTGCTGGCCAGCAAGCGCTTTCCGG |
| | TCGCCACCTTCATCCGCACCCCGGAAGAGCTGGACTAC |
| | CTGCAAGAGCCGGATATCTTCCACGAGATCTTCGGCCA |
| | CTGCCCGCTGCTGACCAATCCCTGGTTCGCCGAATTCA |
| | CCCACACCTACGGCAAGCTCGGCCTGGCCGCGACCAAG |
| | GAACAACGTGTGTACCTGGCACGCTTGTACTGGATGAC |
| | CATCGAGTTTGGCCTGATGGAAACCGCGCAAGGCCGCA |
| | AAATCTATGGTGGTGGCATCCTCTCGTCGCCGAAAGAG |
| | ACCGTCTACAGTCTGTCTGACGAGCCTGAGCACCAGGC |
| | CTTCGACCCGATCGAGGCCATGCGTACACCCTACCGCA |
| | TCGACATTCTGCAACCGGTGTATTTCGTACTGCCGAAC |
| | ATGAAGCGCCTGTTCGACCTGGCCCACGAGGACATCAT |
| | GGGCATGGTCCATAAAGCCATGCAGCTGGGTCTGCATG |
| | CACCGAAGTTTCCACCCAAGGTCGCTGCCTGA-3′ |
| SEQ ID NO: 14 | 5′-ATGAATGCCTTGAACCAAGCCCATTGCGAAGCCTG |
| Nt | CCGCGCCGACGCACCGAAAGTCACCGACGAAGAGCTGG |
| Tetrahydrobiopterin | CCGAGCTGATCCGCGAAATCCCGGACTGGAACATCGAA |
| dehydratase (phhB) | GTACGTGACGGCCACATGGAGCTGGAGCGCGTGTTCCT |
| | GTTCAAGAACTTCAAGCACGCCCTGGCGTTCACCAATG |
| | CCGTGGGCGAAATTGCCGAAGCCGAAGGCCACCACCCA |
| | GGGCTGCTGACTGAATGGGGCAAGGTCACCGTGACCTG |
| | GTGGAGCCACTCGATCAAAGGCCTGCACCGCAACGACT |
| | TCATCATGTGCGCACGCACTGACAAGGTGGCGGAAACG |
| | GCTGAAGGCCGGAAGTAA-3′ |
| SEQ ID NO: 15 | 5′-ATGGCCCCTCGCCCTACCTCACAGAACCAAACCCG |
| Nt | CACATGCCCGACGACGCAGGTTACTCAAGTTGATATAG |
| Tyrosine ammonia | TTGAGAAGATGCTCGCTGCACCAACTGACAGCACCCTA |
| lyase | GAGCTCGACGGGTATTCACTAAATCTTGGGGACGTCGT |
| (TAL_RG_OPT) | TTCAGCTGCAAGGAAAGGAAGACCTGTAAGAGTAAAAG |
| | ATAGTGATGAAATTCGGAGTAAAATAGATAAGTCCGTA |
| | GAGTTTTTAAGGTCACAACTTAGCATGTCCGTATACGG |
| | GGTCACTACCGGGTTCGGCGGTTCCGCCGACACCCGCA |
| | CCGAGGACGCTATATCATTGCAGAAAGCTCTTCTAGAG |
| | CATCAGCTCTGCGGCGTTCTTCCAAGTTCCTTCGATTC |
| | GTTTAGGCTGGGGCGCGGGCTTGAGAACTCTCTGCCCC |
| | TAGAAGTGGTAAGGGGCGCTATGACAATACGGGTGAAC |
| | AGTCTAACAAGAGGTCACAGCGCGGTTAGACTAGTTGT |
| | ACTTGAAGCTCTGACTAACTTCTTAAACCACGGGATTA |
| | CCCCGATTGTCCCACTCCGGGGAACCATCAGTGCGTCC |
| | GGTGACCTATCGCCCCTCTCATATATTGCGGCAGCTAT |
| | ATCAGGACATCCAGATTCAAAGGTTCATGTAGTACATG |
| | AAGGAAAAGAGAAAATACTTTACGCACGCGAGGCCATG |
| | GCCCTTTTTAACCTCGAGCCCGTGGTACTTGGTCCGAA |
| | AGAGGGCCTCGGACTAGTTAACGGTACTGCCGTCAGTG |
| | CCTCAATGGCTACGCTTGCACTCCACGATGCGCACATG |
| | CTGAGCCTGCTAAGTCAAAGTCTCACAGCGATGACCGT |
| | GGAGGCCATGGTGGGGCATGCGGGGTCATTTCATCCAT |
| | TTTTGCATGATGTCACTCGTCCGCATCCTACGCAGATT |
| | GAGGTAGCAGGCAACATTCGCAAGCTTCTCGAGGGAAG |
| | TCGTTTCGCCGTCCATCATGAGGAAGAAGTAAAAGTAA |
| | AGGATGACGAAGGAATATTAAGGCAAGACCGATACCCG |
| | CTCCGCACGTCACCGCAATGGTTGGGTCCACTGGTTTC |
| | AGACCTCATCCACGCACACGCCGTCTTAACTATTGAAG |
| | CAGGGCAATCGACGACAGACAATCCTCTCATCGACGTA |
| | GAGAATAAGACCTCGCATCATGGAGGAAATTTTCAAGC |
| | TGCAGCTGTCGCGAACACAATGGAAAAGACACGTCTCG |
| | GCCTGGCGCAAATAGGGAAACTGAATTTCACCCAGCTC |
| | ACGGAAATGCTGAACGCCGGCATGAACCGCGGCCTGCC |
| | GTCTTGTCTCGCCGCGGAAGATCCTTCTTTATCATATC |
| | ACTGTAAGGGTTTAGATATCGCGGCAGCTGCATATACG |
| | TCCGAACTAGGTCATCTGGCTAACCCTGTCACGACCCA |
| | CGTACAACCGGCGGAGATGGCTAATCAAGCAGTTAACT |
| | CCCTTGCACTAATTTCCGCCCGCCGGACAACAGAGAGT |
| | AACGACGTGTTATCACTGCTGCTCGCTACCCACTTATA |
| | CTGCGTCTTGCAGGCTATCGACTTACGCGCAATCGTGT |
| | TCGAATTTAAGAAGCAATTCGGGCCAGCTATTGTGTCC |
| | CTAATTGATCAGCACTTCGGAAGCGCCATGACTGGGTC |
| | TAATCTTCGAGACGAGCTAGTCGAAAAAGTAAATAAGA |
| | CACTCGCAAAGAGGCTGGAACAGACTAACAGCTACGAC |
| | CTAGTTCCACGGTGGCACGACGCCTTTAGTTTTGCAGC |
| | GGGAACGGTAGTAGAGGTATTGTCATCGACTTCGTTGT |
| | CGTTGGCTGCTGTCAACGCGTGGAAAGTTGCAGCTGCA |
| | GAGTCAGCAATTTCGCTGACGCGGCAAGTACGCGAAAC |
| | ATTTTGGAGCGCTGCTTCGACAAGCTCGCCAGCCCTTT |
| | CTTACCTGTCCCCACGTACGCAGATCTTGTACGCATTC |
| | GTAAGAGAGGAGTTAGGAGTCAAAGCCCGAAGGGGTGA |
| | CGTATTCCTTGGAAAGCAAGAAGTTACAATTGGATCCA |
| | ACGTTTCAAAGATCTATGAGGCCATTAAGAGTGGGCGC |
| | ATAAATAACGTCCTGTTGAAGATGCTGGCCTGA-3′ |
| SEQ ID NO: 16 | 5′-GTGAATAACGAAGCCCGCTCAGGGTCGACCGACCC |
| Nt | TGGCCAACGTCCGCGCTACCGCCAGGTGGCCATCGGGC |
| 4-coumarate-CoA | ATCCCCAGGTGCAGGTCAGTCACGTCGACGACGTGCTG |
| ligase (4-CL) | CGCATGCAACCTGTCGAGCCACTGGCGCCGCTGCCGGC |
| | GCGCCTGCTCGAGCGCCTGGTGCATTGGGCCCAGGTGC |
| | GCCCGGACACCACTTTCATCGCGGCACGCCAGGCAGAC |
| | GGTGCCTGGCGTTCGATCAGCTACGTGCAGATGCTCGC |
| | CGATGTGCGCACCATCGCCGCCAACTTGCTAGGACTGG |
| | GCCTCAGTGCCGAGCGCCCGCTGGCGCTGCTTTCCGGC |
| | AACGACATCGAACACCTGCAAATCGCCCTCGGCGCCAT |
| | GTATGCCGGTATTGCCTATTGCCCGGTGTCGCCGGCCT |
| | ACGCGCTGTTGTCGCAAGACTTCGCCAAGTTGCGCCAT |
| | GTCTGCGAGGTGCTCACCCCCGGAGTGGTCTTCGTCAG |
| | CGACAGCCAGCCGTTCCAGCGCGCCTTCGAGGCGGTGC |
| | TGGACGATTCGGTCGGCGTGATCAGCGTGCGTGGCCAG |
| | GTCGCAGGTCGCCCCCATATAAGCTTCGACAGCCTGTT |
| | GCAACCGGGTGACCTGGCGGCGGCCGATGCGGCTTTCG |
| | CCGCCACCGGGCCGGACACCATCGCCAAATTCCTCTTC |
| | ACCTCGGGCTCGACCAAGCTGCCCAAGGCGGTGATCAC |
| | CACCCAGCGCATGCTGTGCGCCAATCAGCAGATGCTTC |
| | TGCAGACTTTTCCGACGTTCGCCGAGGAGCCGCCGGTG |
| | CTGGTGGACTGGCTGCCGTGGAACCACACGTTCGGCGG |
| | TAGCCACAACCTCGGCATCGTGCTTTACAACGGGGGCA |
| | GTTTCTACCTGGACGCCGGCAAGCCGACCCCGCAAGGC |
| | TTCGCCGAGACCTTGCGCAATCTGCGCGAGATTTCCCC |
| | CACGGCCTACCTCACCGTACCCAAGGGCTGGGAGGAAC |
| | TGGTCAAGGCACTGGAGCAGGACCCCGCGCTACGCGAG |
| | GTGTTCTTTGCCCGCATCAAGCTGTTCTTCTTTGCCGC |
| | CGCAGGCCTGTCGCAAAGCGTCTGGGACCGGCTGGACC |
| | GCATTGCCGAGCAACACTGTGGCGAACGCATCCGCATG |
| | ATGGCCGGCCTTGGCATGACCGAAGCCTCGCCATCGTG |
| | CACCTTCACCACCGGGCCTTTGTCGATGGCCGGCTATG |
| | TCGGGCTGCCGGCACCTGGCTGCGAAGTGAAGCTGGTG |
| | CCGGTGGGCGACAAGCTCGAGGCGCGCTTCCGTGGCCC |
| | GCATATCATGCCGGGCTACTGGCGCTCGCCGCAGCAGA |
| | CCGCCGAGGCGTTCGACGAGGAGGGCTTCTACTGTTCG |
| | GGCGACGCGTTGAAGCTGGCCGATGCCAGGCAGCCCGA |
| | GCTTGGCCTGATGTTCGATGGCCGTATCGCTGAGGACT |
| | TCAAACTTTCGTCCGGGGTATTCGTCAGTGTCGGGCCG |
| | CTGCGCAACCGCGCAGTGCTGGAGGGCTCGCCTTACGT |
| | ACAGGACATCGTGGTCACCGCGCCGGACCGTGAATGCC |
| | TGGGCCTGCTGGTGTTCCCGCGTCTGCCCGAGTGTCGG |
| | CGCCTGGCCGGGCTGGCAGAGGATGCCAGCGATGCGCG |
| | GGTGCTGGCCAACGACACCGTGCGCAGTTGGTTCGCTG |
| | ACTGGCTGGAGCGCTTGAACCGCGATGCCCAAGGCAAC |
| | GCCAGCCGTATCGAATGGCTGTCGCTGCTGGCCGAGCC |
| | GCCGTCGATCGACGCCGGTGAAATCACCGACAAGGGCT |
| | CGATCAATCAGCGCGCCGTGCTGCAGCGGCGCGCCGCT |
| | CAGGTCGAGGCGCTGTACCGTGGCGAAGACCCCGACGC |
| | ATTGCACGCCAAGGTGCGGCCTTGA-3′ |
| SEQ ID NO: 17 | 5′-ATGGCAACTGAGGAGATGAAGAAATTGGCCACCGT |
| Nt | GATGGCCATTGGCACGGCCAACCCTCCGAACTGCTACT |
| Benzalacetone | ACCAGGCCGACTTTCCCGACTTCTACTTCCGCGTCACC |
| synthase (BAS) | AACAGCGACCACCTCATCAACCTCAAGCAAAAGTTCAA |
| | GCGCCTTTGTGAAAACTCAAGGATTGAGAAGCGTTACC |
| | TTCATGTGACCGAAGAGATTCTCAAGGAAAACCCAAAC |
| | ATTGCTGCCTACGAGGCAACCTCGTTGAATGTAAGACA |
| | CAAAATGCAAGTGAAAGGAGTTGCAGAGCTTGGGAAAG |
| | AGGCTGCCCTCAAGGCCATCAAAGAATGGGGCCAACCC |
| | AAGTCCAAGATCACACATCTCATCGTGTGTTGCCTAGC |
| | CGGCGTTGACATGCCCGGCGCGGATTATCAACTCACTA |
| | AGCTTCTTGACCTTGACCCTTCCGTCAAGCGTTTTATG |
| | TTTTACCACCTAGGATGCTACGCTGGTGGCACTGTCCT |
| | TCGCCTTGCAAAGGACATAGCGGAGAACAACAAGGGAG |
| | CTCGTGTTCTCATCGTTTGCTCAGAGATGACAACAACT |
| | TGTTTTCGTGGGCCATCTGAAACCCATCTGGACTCCAT |
| | GATAGGCCAAGCAATATTAGGCGATGGGGCTGCAGCTG |
| | TCATAGTTGGCGCAGATCCAGACCTAACCGTTGAGAGG |
| | CCCATATTCGAGTTGGTTTCCACAGCCCAGACTATTGT |
| | ACCCGAATCCCATGGTGCAATTGAGGGCCACTTGCTTG |
| | AATCTGGACTCAGTTTCCATTTGTACAAGACCGTTCCT |
| | ACACTAATCTCTAACAACATTAAAACTTGCCTTTCTGA |
| | TGCTTTCACTCCTCTAAACATTAGCGATTGGAACTCTC |
| | TTTTCTGGATCGCACACCCTGGTGGTCCTGCCATCCTA |
| | GACCAAGTTACTGCTAAGGTTGGTCTTGAAAAGGAGAA |
| | ACTCAAGGTAACTAGACAAGTGTTGAAGGACTATGGAA |
| | ACATGTCGAGTGCTACGGTGTTTTTCATCATGGATGAG |
| | ATGAGGAAGAAGTCACTCGAAAACGGTCAAGCAACCAC |
| | TGGAGAAGGGCTCGAGTGGGGTGTTTTGTTTGGGTTCG |
| | GGCCTGGAATCACCGTTGAAACTGTAGTGCTACGCAGT |
| | GTGCCCGTAATTAGCTAG-3′ |

### LIST OF CITED DOCUMENTS

### Patent Documents

The following patent document(s) is (are) cited, in case they may be of use:
patcit1: FR1234567 (filing number);
patcit2: US2004230550 (publication number); and
patcit3: FR2795457 (publication number).

### Non-Patent Literature

The following non-patent items are cited, in case they may be of use:
nplcit1: Klesk et al., 2004, J. Agric. Food Chem. 52, 5155-61;
nplcit2: Larsen et al., 1991, Acta Agric. Scand. 41, 447-54).
nplcit3: Kikuchi Y et al. “Mutational analysis of the feedback sites of phenylalanine-sensitive 3-deoxy-d-arabino-heptulosonate-7-phosphate synthase of Escherichia coli.” Appl Environ Microbiol 63:761-762 (1997).): P160L, Q164A, S190A, G191K, S193A, 1225P.
nplcit4: Cui, Di et al., “Molecular basis for feedback inhibition of tyrosine-regulated 3-deoxy-d-arabino-heptulosonate-7-phosphate synthase from Escherichia coli”, J Struct Biol. 2019 Jun. 1; 206(3):322-334.
nplcit5: Lütke-Eversloh and Stephanopoulos, 2005
nplcit6: Lütke-Eversloh and Stephanopoulos, 2007. “L-Tyrosine production by deregulated strains of Escherichia coli”.
nplcit7: Kang et al., 2012 “Artificial biosynthesis of phenylpropanoic acids in a tyrosine overproducing Escherichia coli strain”
nplcit8: Santos et al., 2011. “Optimization of a heterologous pathway for the production of flavonoids from glucose”,
nplcit9: Juminaga et al., 2011. “Modular engineering of L-tyrosine production in E. coli”.
nplcit10: Rodriguez et al., 2015, “Establishment of a yeast platform strain for production of p-coumaric acid through metabolic engineering of aromatic amino acid biosynthesis.”
nplcit11: Calero et al., 2016. “Broad-host range ProUSER vectors enable fast characterization of inducible promoters and optimization of p-coumaric acid production in Pseudomonas putida KT2440”.
nplcit12: Calero et al., 2017. “Genome-wide identification of tolerance mechanisms toward p-coumaric acid in Pseudomonas putida”.
nplcit13: Koeduka, T., et al. (2011). “Characterization of raspberry ketone/zingerone synthase, catalyzing the alpha, beta-hydrogenation of phenylbutenones in raspberry fruits”. Biochemical and Biophysical Research Communications 412, 104-108.
nplcit14: Beekwilder et al., 2007. “Microbial production of natural raspberry ketone”.
nplcit15: Lee et al., 2017. “Heterologous production of raspberry ketone in the wine yeast Saccharomyces cerevisiae via pathway engineering and synthetic enzyme fusion”.
nplcit16: Waterhouse, A. et al., “SWISS-MODEL: homology modelling of protein structures and complexes”. Nucleic Acids Res. 46(W1), W296-W303 (2018)
nplcit17: O. Trott et al., “AutoDock Vina: improving the speed and accuracy of docking with a new scoring function, efficient optimization and multithreading”, Journal of computational chemistry, 2010).
nplcit18: Ding, R. et al. “Introduction of two mutations into AroG increases phenylalanine production in Escherichia coli”. Biotechnol Lett 36, 2103-2108 (2014).
nplcit19: (Gibson et al., 2009).
nplcit20: SchsferA, et al., “Small mobilizable multi-purpose cloning vectors derived from the Escherichia coli plasmids pK18 and pK19: selection of defined deletions in the chromosome of Corynebacterium glutamicum”. Gene 1994 Jul. 22, 145(1): 69-73)
nplcit21: Simon, R., et al. “A broad host range mobilization system for in vivo genetic engineering: transposon mutagenesis in gram negative bacteria”. Nature BioTechnology volume 1, pages784-791 (1983)).
nplcit22: O. Trott et al., 2010. “AutoDock Vina: improving the speed and accuracy of docking with a new scoring function, efficient optimization and multithreading”, Journal of computational chemistry.
nplcit23: Zhou et al., “Characterization of mutants of a tyrosine ammonia-lyase from Rhodotorula glutinis”. Appl. Microbiol. Biotechnol, 2015.
nplcit23: Prior et al., “Broad-host-range vectors for protein expression across gram negative hosts”. Biotechnol Bioeng, 2010 Jun. 1; 106(2): 326-32.

1: A genetically modified strain of Pseudomonas putida, wherein it comprises a mutated AroF-I gene encoding 3-deoxy-D-arabino-heptulosonate-7-phosphate (DAHP) synthase, the sequence of which has at least 90% identity to the sequence SEQ ID NO:1 and having at least one P160L mutation, a P160L/Q164A double mutation, or a P160L/S193A double mutation, preferably a P160L/S193A double mutation.

2: The genetically modified strain of Pseudomonas putida as claimed in claim 1, wherein it is able to express a recombinant DAHP synthase that is resistant to feedback inhibition by tyrosine.

3: The genetically modified strain as claimed in claim 1, wherein it comprises an additional recombinant gene encoding a phenylalanine hydroxylase (phhA), the sequence of which has at least 80% identity to the sequence SEQ ID NO: 5, and an additional recombinant gene encoding a tetrahydrobiopterin dehydratase (phhB), the sequence of which has at least 80% identity to the sequence SEQ ID NO: 6; said recombinant genes phhA and phhB preferably being placed under the control of a heterologous promoter, enabling their overexpression.

4: The genetically modified strain as claimed in claim 1, wherein it expresses a DAHP synthase having an amino acid sequence that has at least 80% identity to the sequence SEQ ID NO: 2, the sequence SEQ ID NO:3, or the sequence SEQ ID NO:4.

5: The genetically modified strain as claimed in claim 4, wherein it expresses a DAHP synthase having an amino acid sequence defined by the sequence SEQ ID NO:2, SEQ ID NO:3, or SEQ ID NO:4.

6: The genetically modified strain as claimed in claim 1, wherein it further comprises one or more additional recombinant genes selected from:
the recombinant gene encoding a polypeptide having tyrosine ammonia lyase (TAL) activity, in particular a TAL polypeptide having at least 80% identity to the amino acid sequence SEQ ID NO:7 of the polypeptide TAL_RG_OPT,
the recombinant gene encoding a polypeptide having 4-coumarate-CoA ligase (4-CL) activity, in particular a 4CL polypeptide having at least 80% identity to the amino acid sequence SEQ ID NO:8, and/or
the recombinant gene encoding a polypeptide having benzalacetone synthase (BAS) activity, in particular a BAS polypeptide having at least 80% identity to the sequence SEQ ID NO:9.

7: The genetically modified strain as claimed in claim 1, wherein it is able to produce a phenylpropanoid compound.

8: A method for synthesizing a phenylpropanoid compound, wherein it comprises a step of growing the genetically modified strain as claimed in claim 7 in a culture medium under conditions that enable the expression of the recombinant genes required for the synthesis of said phenylpropanoid, said phenylpropanoid compound being synthesized by said genetically modified strain.

9: The method as claimed in claim 8, wherein it also comprises a step of recovering the phenylpropanoid compound from the culture medium.

10. (canceled)


SEQUENCE LISTING (17 sequences)

SEQ ID NO: 1 - protein, 370 aa, Pseudomonas putida
LAAGTRDLTSNTMADLPIDDLNVASNETLITPDQLKKEIPLSAKALQTVTAGREVVRNILDGKDHRLFVVVGPCSIHDIKAAHEYAERLKVLAEEVSDTLYLVMRVYFEKPRTTVGWKGLINDPYLDDSFKIQDGLHIGRKLLLDLAEMGLPTATEALDPISPQYLQDLISWSAIGARTTESQTHREMASGLSSAVGFKNGTDGGLTVAINALQSVSKPHRFLGINQEGGVSIVTTKGNPYGHVVLRGGNGKPNYDSVSVALCEQDLAKAKIKANIMVDCSHANSNKDPALQPLVMENVANQILEGNQSIIGLMVESHLNWGCQSIPKNLDDLQYGVSITDACIDWSATEKTLRSMHAKLKDVLPQRKRG

SEQ ID NO: 2 - protein, 370 aa, Pseudomonas putida
LAAGTRDLTSNTMADLPIDDLNVASNETLITPDQLKKEIPLSAKALQTVTAGREVVRNILDGKDHRLFVVVGPCSIHDIKAAHEYAERLKVLAEEVSDTLYLVMRVYFEKPRTTVGWKGLINDPYLDDSFKIQDGLHIGRKLLLDLAEMGLPTATEALDLISPQYLQDLISWSAIGARTTESQTHREMASGLSSAVGFKNGTDGGLTVAINALQSVSKPHRFLGINQEGGVSIVTTKGNPYGHVVLRGGNGKPNYDSVSVALCEQDLAKAKIKANIMVDCSHANSNKDPALQPLVMENVANQILEGNQSIIGLMVESHLNWGCQSIPKNLDDLQYGVSITDACIDWSATEKTLRSMHAKLKDVLPQRKRG

SEQ ID NO: 3 - protein, 740 aa, Pseudomonas putida
LAAGTRDLTSNTMADLPIDDLNVASNETLITPDQLKKEIPLSAKALQTVTAGREVVRNILDGKDHRLFVVVGPCSIHDIKAAHEYAERLKVLAEEVSDTLYLVMRVYFEKPRTTVGWKGLINDPYLDDSFKIQDGLHIGRKLLLDLAEMGLPTATEALDLISPAYLQDLISWSAIGARTTESQTHREMASGLSSAVGFKNGTDGGLTVAINALQSVSKPHRFLGINQEGGVSIVTTKGNPYGHVVLRGGNGKPNYDSVSVALCEQDLAKAKIKANIMVDCSHANSNKDPALQPLVMENVANQILEGNQSIIGLMVESHLNWGCQSIPKNLDDLQYGVSITDACIDWSATEKTLRSMHAKLKDVLPQRKRGLAAGTRDLTSNTMADLPIDDLNVASNETLITPDQLKKEIPLSAKALQTVTAGREVVRNILDGKDHRLFVVVGPCSIHDIKAAHEYAERLKVLAEEVSDTLYLVMRVYFEKPRTTVGWKGLINDPYLDDSFKIQDGLHIGRKLLLDLAEMGLPTATEALDLISPAYLQDLISWSAIGARTTESQTHREMASGLSSAVGFKNGTDGGLTVAINALQSVSKPHRFLGINQEGGVSIVTTKGNPYGHVVLRGGNGKPNYDSVSVALCEQDLAKAKIKANIMVDCSHANSNKDPALQPLVMENVANQILEGNQSIIGLMVESHLNWGCQSIPKNLDDLQYGVSITDACIDWSATEKTLRSMHAKLKDVLPQRKRG

SEQ ID NO: 4 - protein, 370 aa, Pseudomonas putida
LAAGTRDLTSNTMADLPIDDLNVASNETLITPDQLKKEIPLSAKALQTVTAGREVVRNILDGKDHRLFVVVGPCSIHDIKAAHEYAERLKVLAEEVSDTLYLVMRVYFEKPRTTVGWKGLINDPYLDDSFKIQDGLHIGRKLLLDLAEMGLPTATEALDLISPQYLQDLISWSAIGARTTESQTHREMASGLASAVGFKNGTDGGLTVAINALQSVSKPHRFLGINQEGGVSIVTTKGNPYGHVVLRGGNGKPNYDSVSVALCEQDLAKAKIKANIMVDCSHANSNKDPALQPLVMENVANQILEGNQSIIGLMVESHLNWGCQSIPKNLDDLQYGVSITDACIDWSATEKTLRSMHAKLKDVLPQRKRG

SEQ ID NO: 5 - protein, 262 aa, Pseudomonas putida
MKQTQYVAREPDAHGFIDYPQQEHAVWNTLITRQLKVIEGRACQEYLDGIDQLKLPHDRIPQLGEINKVLGATTGWQVARVPALIPFQTFFELLASKRFPVATFIRTPEELDYLQEPDIFHEIFGHCPLLTNPWFAEFTHTYGKLGLAATKEQRVYLARLYWMTIEFGLMETAQGRKIYGGGILSSPKETVYSLSDEPEHQAFDPIEAMRTPYRIDILQPVYFVLPNMKRLFDLAHEDIMGMVHKAMQLGLHAPKFPPKVAA

SEQ ID NO: 6 - protein, 118 aa, Pseudomonas putida
MNALNQAHCEACRADAPKVTDEELAELIREIPDWNIEVRDGHMELERVFLFKNFKHALAFTNAVGEIAEAEGHHPGLLTEWGKVTVTWWSHSIKGLHRNDFIMCARTDKVAETAEGRK

SEQ ID NO: 7 - protein, 693 aa, Rhodotorula glutinis
MAPRPTSQNQTRTCPTTQVTQVDIVEKMLAAPTDSTLELDGYSLNLGDVVSAARKGRPVRVKDSDEIRSKIDKSVEFLRSQLSMSVYGVTTGFGGSADTRTEDAISLQKALLEHQLCGVLPSSFDSFRLGRGLENSLPLEVVRGAMTIRVNSLTRGHSAVRLVVLEALTNFLNHGITPIVPLRGTISASGDLSPLSYIAAAISGHPDSKVHVVHEGKEKILYAREAMALFNLEPVVLGPKEGLGLVNGTAVSASMATLALHDAHMLSLLSQSLTAMTVEAMVGHAGSFHPFLHDVTRPHPTQIEVAGNIRKLLEGSRFAVHHEEEVKVKDDEGILRQDRYPLRTSPQWLGPLVSDLIHAHAVLTIEAGQSTTDNPLIDVENKTSHHGGNFQAAAVANTMEKTRLGLAQIGKLNFTQLTEMLNAGMNRGLPSCLAAEDPSLSYHCKGLDIAAAAYTSELGHLANPVTTHVQPAEMANQAVNSLALISARRTTESNDVLSLLLATHLYCVLQAIDLRAIVFEFKKQFGPAIVSLIDQHFGSAMTGSNLRDELVEKVNKTLAKRLEQTNSYDLVPRWHDAFSFAAGTVVEVLSSTSLSLAAVNAWKVAAAESAISLTRQVRETFWSAASTSSPALSYLSPRTQILYAFVREELGVKARRGDVFLGKQEVTIGSNVSKIYEAIKSGRINNVLLKMLA

SEQ ID NO: 8 - protein, 627 aa, Pseudomonas putida
MNNEARSGSTDPGQRPRYRQVAIGHPQVQVSHVDDVLRMQPVEPLAPLPARLLERLVHWAQVRPDTTFIAARQADGAWRSISYVQMLADVRTIAANLLGLGLSAERPLALLSGNDIEHLQIALGAMYAGIAYCPVSPAYALLSQDFAKLRHVCEVLTPGVVFVSDSQPFQRAFEAVLDDSVGVISVRGQVAGRPHISFDSLLQPGDLAAADAAFAATGPDTIAKFLFTSGSTKLPKAVITTQRMLCANQQMLLQTFPTFAEEPPVLVDWLPWNHTFGGSHNLGIVLYNGGSFYLDAGKPTPQGFAETLRNLREISPTAYLTVPKGWEELVKALEQDPALREVFFARIKLFFFAAAGLSQSVWDRLDRIAEQHCGERIRMMAGLGMTEASPSCTFTTGPLSMAGYVGLPAPGCEVKLVPVGDKLEARFRGPHIMPGYWRSPQQTAEAFDEEGFYCSGDALKLADARQPELGLMFDGRIAEDFKLSSGVFVSVGPLRNRAVLEGSPYVQDIVVTAPDRECLGLLVFPRLPECRRLAGLAEDASDARVLANDTVRSWFADWLERLNRDAQGNASRIEWLSLLAEPPSIDAGEITDKGSINQRAVLQRRAAQVEALYRGEDPDALHAKVRP

SEQ ID NO: 9 - protein, 384 aa, Rheum palmatum
MATEEMKKLATVMAIGTANPPNCYYQADFPDFYFRVTNSDHLINLKQKFKRLCENSRIEKRYLHVTEEILKENPNIAAYEATSLNVRHKMQVKGVAELGKEAALKAIKEWGQPKSKITHLIVCCLAGVDMPGADYQLTKLLDLDPSVKRFMFYHLGCYAGGTVLRLAKDIAENNKGARVLIVCSEMTTTCFRGPSETHLDSMIGQAILGDGAAAVIVGADPDLTVERPIFELVSTAQTIVPESHGAIEGHLLESGLSFHLYKTVPTLISNNIKTCLSDAFTPLNISDWNSLFWIAHPGGPAILDQVTAKVGLEKEKLKVTRQVLKDYGNMSSATVFFIMDEMRKKSLENGQATTGEGLEWGVLFGFGPGITVETVVLRSVPVIS

SEQ ID NO: 10 - protein, 1113 aa, Pseudomonas putida
TTGGCGGCCGGCACCCGTGACCTGACGAGTAACACGATGGCTGATTTACCGATCGATGACTTGAACGTTGCCTCCAACGAGACCCTGATCACCCCTGATCAGCTCAAGAAGGAAATCCCCCTCAGCGCCAAGGCCCTGCAGACCGTGACTGCCGGCCGTGAAGTGGTGCGCAATATTCTCGACGGCAAGGACCATCGCCTGTTCGTCGTGGTCGGCCCTTGCTCCATCCACGACATCAAGGCAGCCCACGAATACGCCGAGCGCCTGAAAGTGCTGGCCGAAGAAGTGTCCGATACGCTGTACCTGGTCATGCGCGTGTACTTCGAAAAGCCGCGCACCACCGTCGGCTGGAAAGGCCTGATCAACGATCCGTACCTGGATGACTCGTTCAAGATCCAGGACGGCCTGCACATCGGCCGCAAGTTGCTGCTGGACCTGGCCGAAATGGGCCTGCCGACCGCCACCGAAGCGCTCGACCTGATTTCGCCGCAGTACCTGCAAGACCTGATCAGCTGGTCGGCCATCGGTGCCCGCACCACCGAATCGCAAACACACCGCGAGATGGCCTCGGGCCTGTCCTCGGCGGTGGGTTTCAAGAACGGTACCGATGGCGGCCTGACCGTTGCCATCAATGCCCTGCAGTCGGTGTCCAAGCCGCACCGCTTCCTGGGCATCAACCAGGAAGGCGGCGTGTCGATCGTCACCACCAAGGGCAACCCATACGGCCACGTGGTACTGCGCGGCGGCAATGGCAAGCCGAACTACGACTCGGTCAGCGTCGCCCTGTGCGAACAGGACCTGGCCAAGGCCAAGATCAAGGCCAACATCATGGTCGACTGCAGCCATGCCAACTCCAACAAGGACCCGGCCCTGCAACCGCTGGTGATGGAGAACGTCGCCAACCAGATTCTCGAAGGCAACCAGTCGATCATCGGCCTGATGGTCGAAAGCCACCTGAACTGGGGCTGTCAGTCCATTCCGAAAAACCTGGACGATTTGCAGTATGGCGTGTCGATCACGGACGCCTGCATCGACTGGTCGGCTACCGAGAAAACCCTGCGCAGCATGCATGCCAAGCTCAAGGATGTGCTGCCGCAGCGTAAGCGCGGCTGA

SEQ ID NO: 11 - protein, 1113 aa, Pseudomonas putida
TTGGCGGCCGGCACCCGTGACCTGACGAGTAACACGATGGCTGATTTACCGATCGATGACTTGAACGTTGCCTCCAACGAGACCCTGATCACCCCTGATCAGCTCAAGAAGGAAATCCCCCTCAGCGCCAAGGCCCTGCAGACCGTGACTGCCGGCCGTGAAGTGGTGCGCAATATTCTCGACGGCAAGGACCATCGCCTGTTCGTCGTGGTCGGCCCTTGCTCCATCCACGACATCAAGGCAGCCCACGAATACGCCGAGCGCCTGAAAGTGCTGGCCGAAGAAGTGTCCGATACGCTGTACCTGGTCATGCGCGTGTACTTCGAAAAGCCGCGCACCACCGTCGGCTGGAAAGGCCTGATCAACGATCCGTACCTGGATGACTCGTTCAAGATCCAGGACGGCCTGCACATCGGCCGCAAGTTGCTGCTGGACCTGGCCGAAATGGGCCTGCCGACCGCCACCGAAGCGCTCGACCTGATTTCGCCGGCCTACCTGCAAGACCTGATCAGCTGGTCGGCCATCGGTGCCCGCACCACCGAATCGCAAACACACCGCGAGATGGCCTCGGGCCTGTCCTCGGCGGTGGGTTTCAAGAACGGTACCGATGGCGGCCTGACCGTTGCCATCAATGCCCTGCAGTCGGTGTCCAAGCCGCACCGCTTCCTGGGCATCAACCAGGAAGGCGGCGTGTCGATCGTCACCACCAAGGGCAACCCATACGGCCACGTGGTACTGCGCGGCGGCAATGGCAAGCCGAACTACGACTCGGTCAGCGTCGCCCTGTGCGAACAGGACCTGGCCAAGGCCAAGATCAAGGCCAACATCATGGTCGACTGCAGCCATGCCAACTCCAACAAGGACCCGGCCCTGCAACCGCTGGTGATGGAGAACGTCGCCAACCAGATTCTCGAAGGCAACCAGTCGATCATCGGCCTGATGGTCGAAAGCCACCTGAACTGGGGCTGTCAGTCCATTCCGAAAAACCTGGACGATTTGCAGTATGGCGTGTCGATCACGGACGCCTGCATCGACTGGTCGGCTACCGAGAAAACCCTGCGCAGCATGCATGCCAAGCTCAAGGATGTGCTGCCGCAGCGTAAGCGCGGCTGA

SEQ ID NO: 12 - protein, 1113 aa, Pseudomonas putida
TTGGCGGCCGGCACCCGTGACCTGACGAGTAACACGATGGCTGATTTACCGATCGATGACTTGAACGTTGCCTCCAACGAGACCCTGATCACCCCTGATCAGCTCAAGAAGGAAATCCCCCTCAGCGCCAAGGCCCTGCAGACCGTGACTGCCGGCCGTGAAGTGGTGCGCAATATTCTCGACGGCAAGGACCATCGCCTGTTCGTCGTGGTCGGCCCTTGCTCCATCCACGACATCAAGGCAGCCCACGAATACGCCGAGCGCCTGAAAGTGCTGGCCGAAGAAGTGTCCGATACGCTGTACCTGGTCATGCGCGTGTACTTCGAAAAGCCGCGCACCACCGTCGGCTGGAAAGGCCTGATCAACGATCCGTACCTGGATGACTCGTTCAAGATCCAGGACGGCCTGCACATCGGCCGCAAGTTGCTGCTGGACCTGGCCGAAATGGGCCTGCCGACCGCCACCGAAGCGCTCGACCTGATTTCGCCGCAGTACCTGCAAGACCTGATCAGCTGGTCGGCCATCGGTGCCCGCACCACCGAATCGCAAACACACCGCGAGATGGCCTCGGGTTTGGCATCGGCGGTGGGTTTCAAGAACGGTACCGATGGCGGCCTGACCGTTGCCATCAATGCCCTGCAGTCGGTGTCCAAGCCGCACCGCTTCCTGGGCATCAACCAGGAAGGCGGCGTGTCGATCGTCACCACCAAGGGCAACCCATACGGCCACGTGGTACTGCGCGGCGGCAATGGCAAGCCGAACTACGACTCGGTCAGCGTCGCCCTGTGCGAACAGGACCTGGCCAAGGCCAAGATCAAGGCCAACATCATGGTCGACTGCAGCCATGCCAACTCCAACAAGGACCCGGCCCTGCAACCGCTGGTGATGGAGAACGTCGCCAACCAGATTCTCGAAGGCAACCAGTCGATCATCGGCCTGATGGTCGAAAGCCACCTGAACTGGGGCTGTCAGTCCATTCCGAAAAACCTGGACGATTTGCAGTATGGCGTGTCGATCACGGACGCCTGCATCGACTGGTCGGCTACCGAGAAAACCCTGCGCAGCATGCATGCCAAGCTCAAGGATGTGCTGCCGCAGCGTAAGCGCGGCTGA

SEQ ID NO: 13 - protein, 789 aa, Pseudomonas putida
ATGAAACAGACGCAATACGTGGCACGCGAGCCCGATGCGCATGGTTTTATCGATTACCCGCAGCAAGAGCATGCGGTGTGGAACACCCTGATCACCCGCCAGCTGAAAGTGATCGAAGGCCGTGCGTGCCAGGAATACCTGGACGGCATCGACCAGCTGAAATTGCCGCATGACCGCATTCCGCAACTGGGCGAGATCAACAAGGTGCTGGGTGCCACCACCGGCTGGCAGGTTGCCCGGGTTCCGGCGCTGATCCCCTTCCAGACCTTCTTCGAATTGCTGGCCAGCAAGCGCTTTCCGGTCGCCACCTTCATCCGCACCCCGGAAGAGCTGGACTACCTGCAAGAGCCGGATATCTTCCACGAGATCTTCGGCCACTGCCCGCTGCTGACCAATCCCTGGTTCGCCGAATTCACCCACACCTACGGCAAGCTCGGCCTGGCCGCGACCAAGGAACAACGTGTGTACCTGGCACGCTTGTACTGGATGACCATCGAGTTTGGCCTGATGGAAACCGCGCAAGGCCGCAAAATCTATGGTGGTGGCATCCTCTCGTCGCCGAAAGAGACCGTCTACAGTCTGTCTGACGAGCCTGAGCACCAGGCCTTCGACCCGATCGAGGCCATGCGTACACCCTACCGCATCGACATTCTGCAACCGGTGTATTTCGTACTGCCGAACATGAAGCGCCTGTTCGACCTGGCCCACGAGGACATCATGGGCATGGTCCATAAAGCCATGCAGCTGGGTCTGCATGCACCGAAGTTTCCACCCAAGGTCGCTGCCTGA

SEQ ID NO: 14 - protein, 357 aa, Pseudomonas putida
ATGAATGCCTTGAACCAAGCCCATTGCGAAGCCTGCCGCGCCGACGCACCGAAAGTCACCGACGAAGAGCTGGCCGAGCTGATCCGCGAAATCCCGGACTGGAACATCGAAGTACGTGACGGCCACATGGAGCTGGAGCGCGTGTTCCTGTTCAAGAACTTCAAGCACGCCCTGGCGTTCACCAATGCCGTGGGCGAAATTGCCGAAGCCGAAGGCCACCACCCAGGGCTGCTGACTGAATGGGGCAAGGTCACCGTGACCTGGTGGAGCCACTCGATCAAAGGCCTGCACCGCAACGACTTCATCATGTGCGCACGCACTGACAAGGTGGCGGAAACGGCTGAAGGCCGGAAGTAA

SEQ ID NO: 15 - protein, 2082 aa, Rhodotorula glutinis
ATGGCCCCTCGCCCTACCTCACAGAACCAAACCCGCACATGCCCGACGACGCAGGTTACTCAAGTTGATATAGTTGAGAAGATGCTCGCTGCACCAACTGACAGCACCCTAGAGCTCGACGGGTATTCACTAAATCTTGGGGACGTCGTTTCAGCTGCAAGGAAAGGAAGACCTGTAAGAGTAAAAGATAGTGATGAAATTCGGAGTAAAATAGATAAGTCCGTAGAGTTTTTAAGGTCACAACTTAGCATGTCCGTATACGGGGTCACTACCGGGTTCGGCGGTTCCGCCGACACCCGCACCGAGGACGCTATATCATTGCAGAAAGCTCTTCTAGAGCATCAGCTCTGCGGCGTTCTTCCAAGTTCCTTCGATTCGTTTAGGCTGGGGCGCGGGCTTGAGAACTCTCTGCCCCTAGAAGTGGTAAGGGGCGCTATGACAATACGGGTGAACAGTCTAACAAGAGGTCACAGCGCGGTTAGACTAGTTGTACTTGAAGCTCTGACTAACTTCTTAAACCACGGGATTACCCCGATTGTCCCACTCCGGGGAACCATCAGTGCGTCCGGTGACCTATCGCCCCTCTCATATATTGCGGCAGCTATATCAGGACATCCAGATTCAAAGGTTCATGTAGTACATGAAGGAAAAGAGAAAATACTTTACGCACGCGAGGCCATGGCCCTTTTTAACCTCGAGCCCGTGGTACTTGGTCCGAAAGAGGGCCTCGGACTAGTTAACGGTACTGCCGTCAGTGCCTCAATGGCTACGCTTGCACTCCACGATGCGCACATGCTGAGCCTGCTAAGTCAAAGTCTCACAGCGATGACCGTGGAGGCCATGGTGGGGCATGCGGGGTCATTTCATCCATTTTTGCATGATGTCACTCGTCCGCATCCTACGCAGATTGAGGTAGCAGGCAACATTCGCAAGCTTCTCGAGGGAAGTCGTTTCGCCGTCCATCATGAGGAAGAAGTAAAAGTAAAGGATGACGAAGGAATATTAAGGCAAGACCGATACCCGCTCCGCACGTCACCGCAATGGTTGGGTCCACTGGTTTCAGACCTCATCCACGCACACGCCGTCTTAACTATTGAAGCAGGGCAATCGACGACAGACAATCCTCTCATCGACGTAGAGAATAAGACCTCGCATCATGGAGGAAATTTTCAAGCTGCAGCTGTCGCGAACACAATGGAAAAGACACGTCTCGGCCTGGCGCAAATAGGGAAACTGAATTTCACCCAGCTCACGGAAATGCTGAACGCCGGCATGAACCGCGGCCTGCCGTCTTGTCTCGCCGCGGAAGATCCTTCTTTATCATATCACTGTAAGGGTTTAGATATCGCGGCAGCTGCATATACGTCCGAACTAGGTCATCTGGCTAACCCTGTCACGACCCACGTACAACCGGCGGAGATGGCTAATCAAGCAGTTAACTCCCTTGCACTAATTTCCGCCCGCCGGACAACAGAGAGTAACGACGTGTTATCACTGCTGCTCGCTACCCACTTATACTGCGTCTTGCAGGCTATCGACTTACGCGCAATCGTGTTCGAATTTAAGAAGCAATTCGGGCCAGCTATTGTGTCCCTAATTGATCAGCACTTCGGAAGCGCCATGACTGGGTCTAATCTTCGAGACGAGCTAGTCGAAAAAGTAAATAAGACACTCGCAAAGAGGCTGGAACAGACTAACAGCTACGACCTAGTTCCACGGTGGCACGACGCCTTTAGTTTTGCAGCGGGAACGGTAGTAGAGGTATTGTCATCGACTTCGTTGTCGTTGGCTGCTGTCAACGCGTGGAAAGTTGCAGCTGCAGAGTCAGCAATTTCGCTGACGCGGCAAGTACGCGAAACATTTTGGAGCGCTGCTTCGACAAGCTCGCCAGCCCTTTCTTACCTGTCCCCACGTACGCAGATCTTGTACGCATTCGTAAGAGAGGAGTTAGGAGTCAAAGCCCGAAGGGGTGACGTATTCCTTGGAAAGCAAGAAGTTACAATTGGATCCAACGTTTCAAAGATCTATGAGGCCATTAAGAGTGGGCGCATAAATAACGTCCTGTTGAAGATGCTGGCCTGA

SEQ ID NO: 16 - protein, 1884 aa, Pseudomonas putida
GTGAATAACGAAGCCCGCTCAGGGTCGACCGACCCTGGCCAACGTCCGCGCTACCGCCAGGTGGCCATCGGGCATCCCCAGGTGCAGGTCAGTCACGTCGACGACGTGCTGCGCATGCAACCTGTCGAGCCACTGGCGCCGCTGCCGGCGCGCCTGCTCGAGCGCCTGGTGCATTGGGCCCAGGTGCGCCCGGACACCACTTTCATCGCGGCACGCCAGGCAGACGGTGCCTGGCGTTCGATCAGCTACGTGCAGATGCTCGCCGATGTGCGCACCATCGCCGCCAACTTGCTAGGACTGGGCCTCAGTGCCGAGCGCCCGCTGGCGCTGCTTTCCGGCAACGACATCGAACACCTGCAAATCGCCCTCGGCGCCATGTATGCCGGTATTGCCTATTGCCCGGTGTCGCCGGCCTACGCGCTGTTGTCGCAAGACTTCGCCAAGTTGCGCCATGTCTGCGAGGTGCTCACCCCCGGAGTGGTCTTCGTCAGCGACAGCCAGCCGTTCCAGCGCGCCTTCGAGGCGGTGCTGGACGATTCGGTCGGCGTGATCAGCGTGCGTGGCCAGGTCGCAGGTCGCCCCCATATAAGCTTCGACAGCCTGTTGCAACCGGGTGACCTGGCGGCGGCCGATGCGGCTTTCGCCGCCACCGGGCCGGACACCATCGCCAAATTCCTCTTCACCTCGGGCTCGACCAAGCTGCCCAAGGCGGTGATCACCACCCAGCGCATGCTGTGCGCCAATCAGCAGATGCTTCTGCAGACTTTTCCGACGTTCGCCGAGGAGCCGCCGGTGCTGGTGGACTGGCTGCCGTGGAACCACACGTTCGGCGGTAGCCACAACCTCGGCATCGTGCTTTACAACGGGGGCAGTTTCTACCTGGACGCCGGCAAGCCGACCCCGCAAGGCTTCGCCGAGACCTTGCGCAATCTGCGCGAGATTTCCCCCACGGCCTACCTCACCGTACCCAAGGGCTGGGAGGAACTGGTCAAGGCACTGGAGCAGGACCCCGCGCTACGCGAGGTGTTCTTTGCCCGCATCAAGCTGTTCTTCTTTGCCGCCGCAGGCCTGTCGCAAAGCGTCTGGGACCGGCTGGACCGCATTGCCGAGCAACACTGTGGCGAACGCATCCGCATGATGGCCGGCCTTGGCATGACCGAAGCCTCGCCATCGTGCACCTTCACCACCGGGCCTTTGTCGATGGCCGGCTATGTCGGGCTGCCGGCACCTGGCTGCGAAGTGAAGCTGGTGCCGGTGGGCGACAAGCTCGAGGCGCGCTTCCGTGGCCCGCATATCATGCCGGGCTACTGGCGCTCGCCGCAGCAGACCGCCGAGGCGTTCGACGAGGAGGGCTTCTACTGTTCGGGCGACGCGTTGAAGCTGGCCGATGCCAGGCAGCCCGAGCTTGGCCTGATGTTCGATGGCCGTATCGCTGAGGACTTCAAACTTTCGTCCGGGGTATTCGTCAGTGTCGGGCCGCTGCGCAACCGCGCAGTGCTGGAGGGCTCGCCTTACGTACAGGACATCGTGGTCACCGCGCCGGACCGTGAATGCCTGGGCCTGCTGGTGTTCCCGCGTCTGCCCGAGTGTCGGCGCCTGGCCGGGCTGGCAGAGGATGCCAGCGATGCGCGGGTGCTGGCCAACGACACCGTGCGCAGTTGGTTCGCTGACTGGCTGGAGCGCTTGAACCGCGATGCCCAAGGCAACGCCAGCCGTATCGAATGGCTGTCGCTGCTGGCCGAGCCGCCGTCGATCGACGCCGGTGAAATCACCGACAAGGGCTCGATCAATCAGCGCGCCGTGCTGCAGCGGCGCGCCGCTCAGGTCGAGGCGCTGTACCGTGGCGAAGACCCCGACGCATTGCACGCCAAGGTGCGGCCTTGA

SEQ ID NO: 17 - protein, 1155 aa, Rheum palmatum
ATGGCAACTGAGGAGATGAAGAAATTGGCCACCGTGATGGCCATTGGCACGGCCAACCCTCCGAACTGCTACTACCAGGCCGACTTTCCCGACTTCTACTTCCGCGTCACCAACAGCGACCACCTCATCAACCTCAAGCAAAAGTTCAAGCGCCTTTGTGAAAACTCAAGGATTGAGAAGCGTTACCTTCATGTGACCGAAGAGATTCTCAAGGAAAACCCAAACATTGCTGCCTACGAGGCAACCTCGTTGAATGTAAGACACAAAATGCAAGTGAAAGGAGTTGCAGAGCTTGGGAAAGAGGCTGCCCTCAAGGCCATCAAAGAATGGGGCCAACCCAAGTCCAAGATCACACATCTCATCGTGTGTTGCCTAGCCGGCGTTGACATGCCCGGCGCGGATTATCAACTCACTAAGCTTCTTGACCTTGACCCTTCCGTCAAGCGTTTTATGTTTTACCACCTAGGATGCTACGCTGGTGGCACTGTCCTTCGCCTTGCAAAGGACATAGCGGAGAACAACAAGGGAGCTCGTGTTCTCATCGTTTGCTCAGAGATGACAACAACTTGTTTTCGTGGGCCATCTGAAACCCATCTGGACTCCATGATAGGCCAAGCAATATTAGGCGATGGGGCTGCAGCTGTCATAGTTGGCGCAGATCCAGACCTAACCGTTGAGAGGCCCATATTCGAGTTGGTTTCCACAGCCCAGACTATTGTACCCGAATCCCATGGTGCAATTGAGGGCCACTTGCTTGAATCTGGACTCAGTTTCCATTTGTACAAGACCGTTCCTACACTAATCTCTAACAACATTAAAACTTGCCTTTCTGATGCTTTCACTCCTCTAAACATTAGCGATTGGAACTCTCTTTTCTGGATCGCACACCCTGGTGGTCCTGCCATCCTAGACCAAGTTACTGCTAAGGTTGGTCTTGAAAAGGAGAAACTCAAGGTAACTAGACAAGTGTTGAAGGACTATGGAAACATGTCGAGTGCTACGGTGTTTTTCATCATGGATGAGATGAGGAAGAAGTCACTCGAAAACGGTCAAGCAACCACTGGAGAAGGGCTCGAGTGGGGTGTTTTGTTTGGGTTCGGGCCTGGAATCACCGTTGAAACTGTAGTGCTACGCAGTGTGCCCGTAATTAGCTAG